(19) United States
(12) Patent Application Publication
(10) Pub. No.: US 2016/0298148 A1
(43) Pub. Date: Oct. 13, 2016

(54) ENRICHMENT OF OILS WITH POLYUNSATURATED FATTY ACIDS

(71) Applicants: Randall WESELAKE, Edmonton (CA); Xue PAN, Edmonton (AB); Rodrigo SILOTO, Edmonton (CA)

(72) Inventors: Randall WESELAKE, Edmonton (CA); Xue PAN, Edmonton (AB); Rodrigo SILOTO, Edmonton (CA)

(21) Appl. No.: 14/405,439

(22) PCT Filed: Jun. 7, 2013
(86) PCT No.: PCT/CA2013/050435

§ 371 Date: Dec. 4, 2014

Related U.S. Application Data
(60) Provisional application No. 61/656,732, filed on Jun. 7, 2012.

(51) Int. Cl. C12P 7/64; C12N 9/10

(52) U.S. Cl. CPC C12P 7/6472; C12N 9/1029; C12Y 203/01158

(57) ABSTRACT

The present invention relates to isolated phospholipid:diacylglycerol acyltransferases (PDAT) and polynucleotide sequences encoding the PDAT enzymes; polynucleotide constructs, vectors and host cells incorporating the polynucleotide sequences; and methods of producing and using same. Also provided are transformed cells and transgenic plants with enhanced oil accumulation and quality.

### FIELD OF THE INVENTION

The present invention relates to novel isolated Linum usitatissimum phospholipid:diacylglycerol acyltransferases (PDATs) and polynucleotide sequences encoding the PDAT enzymes; polynucleotide constructs, vectors, host cells and transgenic organisms incorporating the polynucleotide sequences; and methods of producing and using same.

### BACKGROUND OF THE INVENTION

There is substantial commercial interest in omega-3 polyunsaturated fatty acids (ω-3 PUFAs) due to their wide range of applications. Alpha-linolenic acid (ALA) is an essential omega-3 fatty acid in the diet and the precursor for the omega-3 fatty acid family (Sinclair et al. 2003; Das 206). In addition, with low oxidative stability, ALA can react rapidly with oxygen to polymerize into a soft and durable film upon air exposure, which makes it suitable for domestic and industrial coatings such as varnishes and paints. Omega-3 very long chain PUFAs (ω-3 VLC-PUFA), such as eicosapentaenoic acid (EPA) and docosahexaenoic acid (DHA), are well known for their benefits to human health (Abeywardena & Patten, 2011). Epidemiological, genetic and dietary studies have validated the nutritional value of ω-3 VLC-PUFA in cardiovascular disease (Saravanan et al., 2010) and non-cardiovascular diseases such as rheumatoid arthritis and inflammatory bowel disease (Calder, 2008a, 2008b). Stearidonic acid (SDA), which is generated by the Δ6-desaturation of ALA, exhibits similar health properties (James et al., 2003; Tocher et al., 2006; Harris et al., 2008 and Whelan, 2009). However, natural sources of ω-3 VLC-PUFA and SDA are insufficient for commercial needs.

As a result, numerous attempts have been made to engineer alternative omega-3 PUFA-producing organisms. Genes encoding fatty acid desaturases which directly introduce double bonds into fatty acids have been described (see for example, U.S. Pat. Nos. 5,614,393; 5,952,544; 6,825,335; 7,554,008; 7,671,252; 7,695,950; 7,723,503; Wada et al., 1990). Genetic engineering of VLC-PUFA pathway with various elongases and desaturases in oilseed plants and microorganisms has been successfully demonstrated (see for example, U.S. Pat. Nos. 8,088,974; 7,893,320; 7,736,884; 7,659,120; 7,402,735; 7,364,883; 5,968,809; Ruiz-Lopez et al., 2012). However, the produced lipids contain limited amount of desired PUFAs. Along with elongases and desaturases, many other enzymes can contribute to the flux of PUFAs to storage lipids. Essentially, PUFAs have to be efficiently transferred from the desaturation site (sn-2-phosphatidylcholine, PC) to the substrates for storage lipid synthesis. Therefore, it is also necessary to employ efficient acyltransferases that can enhance the flux of PUFAs from PC to storage lipid.

Triacylglycerols (TAGs) are the main component of seed oil. TAGs can be formed via an acyl-CoA-dependent or acyl-CoA-independent process. The final step of the acyl-CoA dependent pathway, also known as Kennedy pathway (Weiss et al. 1960), is catalyzed by acyl-CoA:diacylglycerol acyltransferase (DGAT) which uses acyl-CoA as acyl donor to convert diacylglycerol to TAG. Phospholipid:diacylglycerol acyltransferase (PDAT) involved in the acyl-CoA independent pathway catalyzes the transfer of a fatty acyl chain from sn-2-phospholipid to sn-1, 2-diacylglycerol to generate TAG (Dahlqvist et al., 2000). Genes encoding PDAT have been reported in yeast (U.S. Pat. No. 7,635,582), microalga Chlamydomonas reinhardtii (Yoon et al. 2012), Arabidopsis thaliana (U.S. Pat. No. 7,635,582), and castor bean (Ricinus communis) (Dahlqvist et al., 2000; Kim et al., 2011; U.S. Pat. No. 8,101,818).

### SUMMARY OF THE INVENTION

The present invention is directed toward the development of oilseeds or oleaginous microorganisms that accumulate oils with enhanced PUFA content by the use of novel PDAT enzymes isolated from Linum usitatissimum. Therefore, in general terms, the present invention relates to isolated LuPDAT1, LuPDAT2, LuPDAT3, LuPDAT4, LuPDAT5, and LuPDAT6 genes from Linum usitatissimum, and methods for their use. The inventors believe that PDAT enzymes utilize preferentially substrates containing ALA in flax. In addition, the inventors believe that the substrate selectivity of the identified novel PDAT5 is not limited to ALA, but extended to other PUFAs, including SDA, γ-linolenic acid (GLA) and EPA. Accordingly, these PDAT enzymes may be used in recombinant methods to engineer production of PUFA enriched products.

In one aspect, the invention comprises an isolated polynucleotide sequence encoding a protein or polypeptide comprising or consisting of an amino acid sequence selected from the group consisting of SEQ ID NOS: 7, 8, 9, 10, 11, or 12, respective biologically active variants and biologically active portions thereof, with respective sequences having at least 80%, 85%, 90%, or 95% identity, and wherein the variants have PDAT activity.

In one embodiment, the invention includes an isolated polynucleotide encodes a polypeptide having PDAT activity, wherein the polypeptide comprises the amino acid sequence of at least 80%, 85%, 90% or 95% sequence identity based on the Clustal W method of alignment when compared to one of SEQ ID NOS: 7, 8, 9, 10, 11 or 12.

In one embodiment, the polynucleotide comprises the nucleotide sequence of one of SEQ ID NO: 1, 2, 3, 4, 5 or 6.

In another aspect, the invention comprises a recombinant expression vector comprising at least one polynucleotide as described herein, operably linked with transcriptional and translational regulatory regions or sequences to provide for expression of the at least one polynucleotide sequence, expressible in bacterial, yeast, fungal, mammalian or plant cells.

In another aspect, the invention comprises a microbial cell comprising the above recombinant expression vector. In one embodiment, the cell comprises Saccharomyces cerevisiae and which comprises a recombinant expression vector comprising the sequence of any of the isolated polynucleotides of the present invention. The cell may be engineered to have reduced TAG synthesis ability. In one embodiment, the cell may further comprise a recombinant expression vector expressing a non-native elongase or desaturase enzyme, or both.

In another aspect, the invention comprises a method for producing TAG with enriched PUFA content in oleaginous microbial cells comprising the steps of:

a) transforming a host microbial cell with a recombinant expression vector comprising one of SEQ ID NO: 7, 8, 10 or 11 under conditions sufficient for expression of a PDAT polynucleotide; and

b) exposing the host microbial cell to certain PUFA, wherein the PUFA is converted by the PDAT into TAO.

The exposure to certain PUPA may be accomplished by providing the PUFA exogenously, or engineering the cell to produce, or preferentially produce the certain PUFA endogenously.

In one embodiment, the fatty acid substrate comprises one or more of ALA, GLA, SDA, and EPA.

In another aspect, the invention comprises a transgenic plant, plant cell, plant seed, callus, plant embryo, microspore-derived embryo, or microspore, comprising the above recombinant expression vector. In one embodiment, the transgenic plant, plant cell, plant seed, callus, plant embryo, microspore-derived embryo, or microspore is selected from a linseed, rapeseed, canola, peanut, safflower, flax, hemp, camelina, soybean, pea, sunflower, olive, palm, oats, wheat, triticale, barley, corn, and legume plant, plant cell, plant seed, callus, plant embryo, or microspore-derived embryo or microspore.

In another aspect, the invention comprises a method for producing a transgenic plant comprising the steps of introducing into a plant cell or a plant tissue the above recombinant expression vector to produce a transformed cell or plant tissue; and cultivating the transformed plant cell or transformed plant tissue to produce the transgenic plant. In one embodiment, the plant is selected from a linseed, rapeseed, canola, peanut, safflower, flax, hemp, camelina, soybean, pea, sunflower, olive, palm, oats, wheat, triticale, barley, corn, and legume plant, plant cell, plant seed, callus, plant embryo, or microspore-derived embryo or microspore. In one embodiment, the transgenic plant has or is engineered to have reduced DGAT activity compared to a non-transgenic plant, and accumulates fatty acids including ALA, SDA, and EPA with the exception of GLA.

Additional aspects and advantages of the present invention will be apparent in view of the description, which follows. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The invention will now be described by way of an exemplary embodiment with reference to the accompanying simplified, diagrammatic, not-to-scale drawings.

FIG. 1. Shows a phylogenetic tree of the amino acid sequences of LuPDATs isolated from flax. Six flax PDATs can be divided into three branches: I (LuPDAT1 and LuPDAT5), II (LuPDAT2 and LuPDAT4) and III (LuPDAT3 and LuPDAT6). The multiple sequence alignments of LuPDATs were generated using the Clustal W module within MEGA5 with the defaul parameters (gap penalty, 10.0; gap length penalty, 0.2; Gonnet matrix). The phylogenetic trees were constructed using the same software with the following parameters: neighbor-joining method, Poisson model, complete deletion and bootstrap (1000 replicates). Numbers above branches indicate the percentage of bootstrap values.

FIG. 2. The α-linolenic acid (ALA)-specific activity of LuPDAT1 and LuPDAT2 in yeast strain H1246. Yeast transformed with pYESlacZ was used as the negative control, which was annotated as “LacZ” on the TLC plate. The square bracket indicates the position of triacylglycerol (TAG) produced by the yeast cells. The corresponding fatty acid used for feeding is shown on the left of the figure with the chemical structure (OA, oleic acid; LA, linoleic acid; ALA). All fatty acids were provided at concentration of 100 μM. P1—LuPDAT1 (SEQ ID NO: 7); P2—LuPDAT2 (SEQ ID NO: 8); P6—LuPDAT6 (SEQ ID NO: 9); TAG—triolein TAG standards.

FIG. 3. The polyunsaturated fatty acid (PUFA)-specific activity of LuPDAT1 and LuPDAT2 in yeast strain H1246. Yeast transformed with pYESlacZ was used as the negative control, which was annotated as “−” on the TLC plate. The corresponding fatty acid used for feeding is shown on the left of the figure with the chemical structure. All fatty acids were provided at concentration of 100 μM. P1—LuPDAT1 (SEQ ID NO: 7); P2—LuPDAT2 (SEQ ID NO: 8); P6—LuPDAT6 (SEQ ID NO: 9); TAG—trilinolenin TAG standards; SDA—stearidonic acid; GLA—γ-linolenic acid; DGLA—dihomo-γ-linolenic acid; AA—arachidonic acid; EPA—eicosapentaenoic acid; ETA—eicosatrienoic acid; DHA—docosahexaenoic acid.

FIG. 4. Gas chromatography-mass spectrometry (GC-MS) chromatograms of yeast strain H1246 expressing LuPDAT1 and LuPDAT2 in the presence of α-linolenic acid (ALA). Yeast cells expressing LuPDAT1 or LuPDAT2 is capable of producing triacylglycerol (TAG) containing only ALA (trilinolenin). The recombinant yeast cells were cultivated in the presence of 100 μM of ALA. The yeast lipids were extracted and separated by thin layer chromatography (TLC) plate. The compound corresponding to the upper and lower TAG bands (marked by square brackets) was scrapped separately from the TLC plate, transmethylated and analyzed through GC-MS. The ratio of TAG within each separated band to the total amount of TAG was calculated and the values are shown as percentage on the upper left corner of each chromatograph. P1—LuPDAT1; P2—LuPDAT2.

FIG. 5. The concentration effect of the exogenously provided α-linolenic acid (ALA) on overall percentage of ALA in triacylglycerol (TAG) and total TAG content in yeast strain H1246 expressing LuPDAT1 or LuPDAT2. FIG. 5A shows that yeast cells expressing LuPDAT1 or LuPDAT2 are capable of producing TAG with up to 90% ALA. In addition, FIG. 5B shows that an increased concentration of supplemented ALA from 0 to 300 μM leads to approximately 168-fold and 44-fold increases in total TAG content on a dry weight basis for yeast expressing LuPDAT1 and LuPDAT2, respectively. The yeast cells were cultivated in the absence or presence of different ALA concentration and harvested at the same growth stage (OD_{600 nm}=6.5±0.05). The collected samples were first freeze-dried for 16 h and then subjected to lipid extraction and gas chromatography-mass spectrometry (GC-MS) analysis for the determination of total TAG content and fatty acid composition. Data are presented as means±SE (n=4).

FIG. 6. The fatty acid methyl esters (FAME) profile of yeast strain H1246 co-expressing LuPDAT1 and LuPDAT2 individually with desaturases. Co-expression of either LuPDAT1 (black bar) or LuPDAT2 (grey bar) with LuFAD2-1 and LuFAD3B in yeast produces triacylglycerol (TAG) with α-linolenic acid (ALA, C 18:3) as the predominant fatty acid. Yeast cultures were induced at 20° C. for three days before harvested. Data are presented as means±SE (n=3).

FIG. 7. Fatty acid composition of LuPDAT-overexpressing seeds. Overexpression of LuPDAT1 and LuPDAT2 in wild-type Arabidopsis seeds results in an increased level of linoleic acid (LA) and α-linolenic acid (ALA). Ten individual transgenic lines were analyzed for each construct. Wild-type Arabidopsis transformed with empty pGreen plasmid were used as controls (CTR). Data are presented as means±SE (n=3), with asterisks indicating p<0.05 (ANOVA, Duncan's multiple range test). OA—oleic acid; EA—eicosenoic acid.

FIG. 8. The LuPDAT1 nucleotide sequence.

FIG. 9. The LuPDAT2 nucleotide sequence.

FIG. 10. The LuPDAT3 nucleotide sequence.

FIG. 11. The LuPDAT4 nucleotide sequence.

FIG. 12. The LuPDAT5 nucleotide sequence.

FIG. 13. The LuPDAT6 nucleotide sequence.

FIG. 14. The LuPDAT1 amino acid sequence.

FIG. 15. The LuPDAT2 amino acid sequence.

FIG. 16. The LuPDAT3 amino acid sequence.

FIG. 17. The LuPDAT4 amino acid sequence.

FIG. 18. The LuPDAT5 amino acid sequence.

FIG. 19. The LuPDAT6 amino acid sequence.

FIGS. 20A-C show an amino acid alignment of the polypeptides LuPDAT1 (SEQ ID NO: 7), LuPDAT2 (SEQ ID NO: 8), LuPDAT3 (SEQ ID NO: 9), LuPDAT4 (SEQ ID NO: 10), LuPDAT5 (SEQ ID NO: 11) and LuPDAT6 (SEQ ID NO: 12).

### DETAILED DESCRIPTION OF PREFERRED EMBODIMENTS

The present invention relates to isolated polynucleotides of the LuPDAT1, LuPDAT2, LuPDAT3, LuPDAT4, LuPDAT5, and LuPDAT6 genes from Linum usitatissimum; nucleic acid constructs, recombinant expression vectors and host cells incorporating the polynucleotide sequences; and methods of producing and using same. When describing the present invention, all terms not defined herein have their common art-recognized meanings. To the extent that the following description is of a specific embodiment or a particular use of the invention, it is intended to be illustrative only, and not limiting of the claimed invention. The following description is intended to cover all alternatives, modifications and equivalents that are included in the spirit and scope of the invention, as defined in the appended claims.

To facilitate understanding of the invention, the following definitions are provided.

A “cDNA” is a polynucleotide which is complementary to a molecule of mRNA. The “cDNA” is formed of a coding sequence flanked by 5′ and 3′ untranslated sequences. A “coding sequence” or “coding region” or “open reading frame (ORF)” is part of a gene that codes for an amino acid sequence of a polypeptide.

A “complementary sequence” is a sequence of nucleotides which forms a duplex with another sequence of nucleotides according to Watson-Crick base pairing rules where “A” pairs with “T” and “C” pairs with “G.”

A “construct” is a polynucleotide which is formed by polynucleotide segments isolated from a naturally occurring gene or which is chemically synthesized. The “construct” which is combined in a manner that otherwise would not exist in nature, is usually made to achieve certain purposes. For instance, the coding region from “gene A” can be combined with an inducible promoter from “gene B”, so the expression of the recombinant construct can be induced.

“Downstream” means on the 3′ side of a polynucleotide while “upstream” means on the 5′ side of a polynucleotide.

“Expression” refers to the transcription of a gene into RNA (rRNA, tRNA) or messenger RNA (mRNA) with subsequent translation into a protein.

“Gene” means a DNA segment which contributes to phenotype or function, and which may be characterized by sequence, transcription or homology.

“Isolated” means that a substance or a group of substances is removed from the coexisting materials of its natural state.

“Nucleic acid” means polynucleotides such as deoxyribonucleic acid (DNA), and, where appropriate, ribonucleic acid (RNA). The term should also be understood to include, as equivalents, analogs of either RNA or DNA.

As used herein, the term “plasmid” means a DNA molecule which is separate from, and can replicate independently of, the chromosomal DNA. They are double stranded and, in many cases, circular. Plasmids used in genetic engineering are known as vectors and are used to multiply or express particular genes. Any plasmid may be used for the present invention provided that the plasmid contains a gene which encodes a LuPDAT1, LuPDAT2, LuPDAT3, LuPDAT4, LuPDAT5, and LuPDAT6, or a variant thereof in an expressible manner. In one embodiment, the plasmid comprises a yeast expression vector. Those skilled in art will recognize that any plasmid in the art may be modified for use in the compositions and methods of the present invention. As used herein, the term “regulatory element” includes, but is not limited to, a promoter, enhancer, terminator, and the like which are required for the expression of the encoded LuPDAT1, LuPDAT2, LuPDAT3, LuPDAT4, LuPDAT5, and LuPDAT6, or variant thereof.

A “polynucleotide” is a linear sequence of ribonucleotides (RNA) or deoxyribonucleotides (DNA) in which the 3′ carbon of the pentose sugar of one nucleotide is linked to the 5′ carbon of the pentose sugar of another nucleotide. The deoxyribonucleotide bases are abbreviated as “A” deoxyadenine; “C” deoxycytidine; “G” deoxyguanine; “T” deoxythymidine; “I” deoxyinosine. Some oligonucleotides described herein are produced synthetically and contain different deoxyribonucleotides occupying the same position in the sequence. The blends of deoxyribonucleotides are abbreviated as “W” A or T; “Y” C or T; “H” A, C or T; “K” G or T; “D” A, G or T; “B” C, G or T; “N” A, C, G or T.

A “polypeptide” is a linear sequence of amino acids linked by peptide bonds. The amino acids are abbreviated as “A” alanine; “R” arginine; “N” asparagine; “D” aspartic acid; “C” cysteine; “Q” glutamine; “E” glutamic acid; “G” glycine; “H” histidine; “I” isoleucine; “L” leucine; “K” lysine; “M” methionine; “F” phenylalanine; “P” proline; “S” serine; “T” threonine; “W” tryptophan; “Y” tyrosine and “V” valine.

Two polynucleotides or polypeptides are “identical” if the sequence of nucleotides or amino acids, respectively, in the two sequences is the same when aligned for maximum correspondence as described here. Sequence comparisons between two or more polynucleotides or polypeptides can be generally performed by comparing portions of the two sequences over a comparison window which can be from about 20 to about 200 nucleotides or amino acids, or more. The “percentage of sequence identity” may be determined by comparing two optimally aligned sequences over a comparison window, wherein the portion of a polynucleotide or a polypeptide sequence may include additions (i.e., insertions) or deletions (i.e., gaps) as compared to the reference sequence. The percentage is calculated by determining the positions at which identical nucleotides or identical amino acids are present, dividing by the number of positions in the window and multiplying the result by 100 to yield the percentage of sequence identity. Polynucleotide and polypeptide sequence alignment may be performed by implementing specialized algorithms or by inspection. Examples of sequence comparison and multiple sequence alignment algorithms are: BLAST and ClustalW software. Identity between nucleotide sequences can also be determined by DNA hybridization analysis, wherein the stability of the double-stranded DNA hybrid is dependent on the extent of base pairing that occurs. Conditions of high temperature and/or low salt content reduce the stability of the hybrid, and can be varied to prevent annealing of sequences having less than a selected degree of homology. Hybridization methods are described in Ausubel et al. (2000).

A “fatty acid” is a carboxylic acid having an unbranched aliphatic chain.

A “polyunsaturated fatty acid” (PUFA) is a fatty acid with more than one carbon-carbon double bond.

A “long chain polyunsaturated fatty acid” (LC-PUFA) is a fatty acid with a chain of 18 or more carbons atoms and three or more double bonds in the cis configuration.

A “very long chain polyunsaturated fatty acid” (VLC-PUFA) is a fatty acid with a chain of 20 or more carbons atoms and three or more double bonds in the cis configuration.

An “omega-3 fatty acid” is a polyunsaturated fatty acid with the first double bond beginning at the third carbon from the methyl end of the carbon chain. An “omega-6 fatty acid” is a polyunsaturated fatty acid with the first double bond at the sixth carbon counting from the methyl end of the carbon chain.

A “triacylglycerol” is an ester having three fatty carboxylic acids attached to a single glycerol backbone. It is the main component of vegetable oil and animal fats. Alternative names include: triglyceride, triacylglyceride, TG and TAG.

A “phospholipid:diacylglycerol acyl transferase” (PDAT) is an enzyme of the class EC 2.3.1.158 which catalyzes the reaction: phospholipid+1,2-diacylglycerol⇄lysophospholipid+TAG. A “LuPDAT” is a gene encoding a PDAT from flax. A number denoted after LuPDAT (for example, LuPDAT1) refers to a specific gene encoding a PDAT. A “LuPDAT” refers to a polypeptide from flax which exhibits PDAT enzymatic activity. A number denoted after LuPDAT (for example, LuPDAT1) refers to a specific polypeptide which exhibits PDAT enzyme activity. A polypeptide having “PDAT activity” is a polypeptide that has, to a greater or lesser degree, the enzymatic activity of PDAT.

A “promoter” is a polynucleotide usually located within 20 to 5000 nucleotides upstream of the initiation of translation site of a gene. The “promoter” determines the first step of expression by providing a binding site to DNA polymerase to initiate the transcription of a gene. The promoter is said to be “inducible” when the initiation of transcription occurs only when a specific agent or chemical substance is presented to the cell. For instance, the GAL “promoter” from yeast is “inducible by galactose,” meaning that this GAL promoter allows initiation of transcription and subsequent expression only when galactose is presented to yeast cells.

“Transformation” means the directed modification of the genome of a cell by external application of a polynucleotide, for instance, a construct. The inserted polynucleotide may or may not integrate with the host cell chromosome. For example, in bacteria, the inserted polynucleotide usually does not integrate with the bacterial genome and might replicate autonomously. In plants, the inserted polynucleotide integrates with the plant chromosome and replicates together with the plant chromatin.

A “transgenic” organism is the organism that was transformed with an external polynucleotide. The “transgenic” organism encompasses all descendants, hybrids and crosses thereof, whether reproduced sexually or asexually and which continue to harbor the foreign polynucleotide.

A “vector” is a polynucleotide that is able to replicate autonomously in a host cell and is able to accept other polynucleotides. For autonomous replication, the vector contains an “origin of replication.” The vector usually contains a “selectable marker” that confers the host cell resistance to certain environment and growth conditions. For instance, a vector that is used to transform bacteria usually contains a certain antibiotic “selectable marker” which confers the transformed bacteria resistance to such antibiotic.

The present invention relates to isolated polynucleotides and polypeptides of the LuPDAT1, LuPDAT2, LuPDAT3, LuPDAT4, LuPDAT5, or LuPDAT6 genes from Linum usitatissimum; nucleic acid constructs, vectors, host cells and transgenic organisms incorporating the polynucleotide sequences; and methods of producing and using same.

In one aspect, the invention provides isolated LuPDAT1, LuPDAT2, LuPDAT3, LuPDAT4, LuPDAT5, or LuPDAT6 polynucleotides, and polypeptides having PDAT activity. LuPDAT1, LuPDAT2, LuPDAT3, LuPDAT4, LuPDAT5, and LuPDAT6 polynucleotides include, without limitation (1) single- or double-stranded DNA, such as cDNA or genomic DNA including sense and antisense strands; and (2) RNA, such as mRNA. LuPDAT1, LuPDAT2, LuPDAT3, LuPDAT4, LuPDAT5, and LuPDAT6 polynucleotides include at least a coding sequence which codes for the amino acid sequence of the specified PDAT polypeptide, but may also include 5′ or 3′ untranslated regions and transcriptional regulatory elements such as promoters and enhancers found upstream or downstream from the transcribed region.

In one embodiment, the invention provides a LuPDAT1 polynucleotide which is a cDNA comprising the nucleotide sequence depicted in SEQ ID NO: 1, and which was isolated from Linum usitatissimum. The cDNA comprises a coding region of 2088 base pairs. The LuPDAT1 encoded by the coding region (designated as LuPDAT1, SEQ ID NO: 7) is a 695 amino acid polypeptide with a predicted molecular weight of 76,891.89 Daltons and an isoelectric point of 8.23. LuPDAT1 exhibits a preference for fatty acid substrates comprising ALA, GLA, SDA and EPA.

In one embodiment, the invention provides a LuPDAT2 polynucleotide which is a cDNA comprising the nucleotide sequence depicted in SEQ ID NO: 2, and which was isolated from Linum usitatissimum. The cDNA comprises a coding region of 2145 base pairs. The LuPDAT2 encoded by the coding region (designated as LuPADT2, SEQ ID NO: 8) is a 714 amino acid polypeptide with a predicted molecular weight of 79,072.61 Daltons and an isoelectric point of 6.4. LuPDAT2 exhibits a preference for fatty acid substrates comprising ALA, GLA, SDA and EPA.

In one embodiment, the invention provides a LuPDAT3 polynucleotide which is a cDNA comprising the nucleotide sequence depicted in SEQ ID NO: 3, and which was isolated from Linum usitatissimum. The cDNA comprises a coding region of 1728 base pairs. The LuPDAT3 encoded by the coding region (designated as LuPDAT3, SEQ ID NO: 9) is a 575 amino acid polypeptide with a predicted molecular weight of 63,093.05 Daltons and an isoelectric point of 6.19.

In one embodiment, the invention provides a LuPDAT4 polynucleotide which is a cDNA comprising the nucleotide sequence depicted in SEQ ID NO: 4, and which was isolated from Linum usitatissimum. The cDNA comprises a coding region of 2148 base pairs. The LuPDAT4 encoded by the coding region (designated as LuPDAT4, SEQ ID NO: 10) is a 715amino acid polypeptide with a predicted molecular weight of 78,923.51 Daltons and an isoelectric point of 6.72. LuPDAT4 exhibits a preference for fatty acid substrates comprising ALA, GLA, SDA and EPA.

In one embodiment, the invention provides a LuPDAT5 polynucleotide which is a cDNA comprising the nucleotide sequence depicted in SEQ ID NO: 5, and which was isolated from Linum usitatissimum. The cDNA comprises a coding region of 2088 base pairs. The LuPDAT5 encoded by the coding region (designated as LuPDAT5, SEQ ID NO: 11) is a 695 amino acid polypeptide with a predicted molecular weight of 76,807.6 Daltons and an isoelectric point of 8.28. LuPDAT5 exhibits a preference for fatty acid substrates comprising ALA, GLA, SDA and EPA.

In one embodiment, the invention provides a LuPDAT6 polynucleotide which is a cDNA comprising the nucleotide sequence depicted in SEQ ID NO: 6, and which was isolated from Linum usitatissimum. The cDNA comprises a coding region of 1719 base pairs. The LuPDAT6 encoded by the coding region (designated as LuPDAT6, SEQ ID NO: 12) is a 572 amino acid polypeptide with a predicted molecular weight of 62,792.54 Daltons and an isoelectric point of 6.19.

Those skilled in the art will recognize that the degeneracy of the genetic code allows for a plurality of polynucleotides to encode for identical polypeptides. Accordingly, the invention includes polynucleotides of SEQ ID NOS: 1-6, and variants of these polynucleotides which encode the polypeptides of SEQ ID NOS: 7-12. In one embodiment, polynucleotides having at least 80%, at least 85%, at least 90%, at least 95%, at least 96%, at least 97%, at least 98%, or at least 99% sequence identity to the nucleotide sequences depicted in SEQ ID NOS: 1-6 are included in the invention. Methods for isolation of such polynucleotides are well known in the art (see for example, Ausubel et al., 2000).

In one embodiment, the invention provides isolated polynucleotides which encode LuPDAT1, LuPDAT2, LuPDAT3, LuPDAT4, LuPDAT5, and LuPDAT6, or polypeptides having amino acid sequences having at least 80%, 85%, at least 90%, at least 95%, at least 96%, at least 97%, at least 98%, or at least 99% sequence identity to the amino acid sequences depicted in SEQ ID NOS: 7-12.

The above described polynucleotides of the invention may be used to express polypeptides in recombinantly engineered cells including, for example, bacterial, yeast, fungal, mammalian or plant cells. In one embodiment, the invention provides polynucleotide constructs, vectors and cells comprising LuPDAT1, LuPDAT2, LuPDAT3, LuPDAT4, LuPDAT5, and LuPDAT6 polynucleotides. Those skilled in the art are knowledgeable in the numerous systems available for expression of a polynucleotide. All systems employ a similar approach, whereby an expression construct is assembled to include the protein coding sequence of interest and control sequences such as promoters, enhancers, and terminators, with signal sequences and selectable markers included if desired. Briefly, the expression of isolated polynucleotides encoding polypeptides is typically achieved by operably linking, for example, the DNA or cDNA to a constitutive or inducible promoter, followed by incorporation into an expression vector. The vectors can be suitable for replication and integration in either prokaryotes or eukaryotes. Typical expression vectors include transcription and translation terminators, initiation sequences, and promoters useful for regulation of the expression of the DNA. High-level expression of a cloned gene is obtained by constructing expression vectors which contain a strong promoter to direct transcription, a ribosome binding site for translational initiation, and a transcription/translation terminator. Vectors may further comprise transit and targeting sequences, selectable markers, enhancers or operators. Means for preparing vectors are well known in the art. Typical vectors useful for expression of polynucleotides in plants include for example, vectors derived from the Ti plasmid of Agrobacterium tumefaciens and the pCaM-VCN transfer control vector. Promoters suitable for plant cells include for example, the nopaline synthase, octopine synthase, and mannopine synthase promoters, and the caulimovirus promoters (Bevan et al., 1986). Seed-specific promoters, such as ACP and napin-derived transcription initiation regions, have been shown to confer preferential expression of a specific gene in plant seed tissue (Breen & Crouch, 1992; Okamuro & Goldberg, 1989). In one embodiment, the seed-specific napin promoter is preferred.

Those skilled in the art will appreciate that modifications (i.e., amino acid substitutions, additions, deletions and post-translational modifications) can be made to a polypeptide of the invention without eliminating or diminishing its biological activity. Conservative amino acid substitutions (i.e., substitution of one amino acid for another amino acid of similar size, charge, polarity and conformation) or substitution of one amino acid for another within the same group (i.e., nonpolar group, polar group, positively charged group, negatively charged group) are unlikely to alter protein function adversely. Some modifications may be made to facilitate the cloning, expression or purification. Variant LuPDAT1, LuPDAT2, LuPDAT3, LuPDAT4, LuPDAT5, and LuPDAT6 polypeptides may be obtained by mutagenesis of the polynucleotides depicted in SEQ ID NOS: 1-6 using techniques known in the art including, for example, oligonucleotide-directed mutagenesis, region-specific mutagenesis, linker-scanning mutagenesis, and site-directed mutagenesis by PCR (Ausubel et al., 2000).

Various methods for transformation or transfection of cells are available. For prokaryotes, lower eukaryotes and animal cells, such methods include for example, calcium phosphate precipitation, fusion of the recipient cells with bacterial protoplasts containing the DNA, treatment of the recipient cells with liposomes containing the DNA, DEAE dextran, electroporation, biolistics and microinjection. Various industrial strains of microorganisms including for example, fungi, such as Mortierella or Traustochytrium; mosses such as Physcomitrella or Ceratodon; algae such as Crypthecodinium or Phaeodactylum; or Aspergillus, Pichia pastoris, Saccharomyces cerevisiae may be used to express LuPDAT1, LuPDAT2, LuPDAT3, LuPDAT4, LuPDAT5, and LuPDAT6 polypeptides. The LiAc/ssDNA/PEG yeast transformation method is most efficient procedure for introducing a recombinant DNA construct into yeast cell. Alternatively, exogenous DNA may be transferred into yeast by electroporation, biolistics, glass bead agitation and spheroplasts (Gietz & Woods, 2001). In one embodiment, the LiAc/ssDNA/PEG method is conducted to introduce LuPDAT1, LuPDAT2, LuPDAT3, LuPDAT4, LuPDAT5 or LuPDAT6 polynucleotides into yeast cells.

Methods for transformation of plant cells include for example, infiltration, electroporation, PEG poration, particle bombardment, Agrobacterium tumefaciens- or Agrobacterium rhizogenes-mediated transformation, direct protoplast transformation, and microinjection (Rakoczy-Trojanowska, 2002). The transformed plant cells, seeds, callus, embryos, microspore-derived embryos, microspores, organs or explants are cultured or cultivated using standard plant tissue culture techniques and growth media to regenerate a whole transgenic plant which possesses the transformed genotype. Transformation may be confirmed by use of a DNA marker gene encoding for an enzyme that confers herbicide tolerance (Block et al., 1987) or antibiotic resistance; catalyzes deamination of D-amino acids (Erikson et al., 2004); or by conducting methods such as PCR or Southern blot hybridization (Ausubel et al., 2000; Sambrook et al., 1989). Transgenic plants may pass polynucleotides encoding LuPDAT1, LuPDAT2, LuPDAT3, LuPDAT4, LuPDAT5, and LuPDAT6 polypeptides to their progeny, or can be further crossbred with other species. Accordingly, in one embodiment, the invention provides methods for producing transgenic plants, plant cells, callus, seeds, plant embryos, microspore-derived embryos, and microspores comprising LuPDAT1, LuPDAT2, LuPDAT3, LuPDAT4, LuPDAT5, and LuPDAT6 polynucleotides.

In one embodiment, the invention provides transgenic plants, plant cells, callus, seeds, plant embryos, microspore-derived embryos, or microspores, comprising LuPDAT1, LuPDAT2, LuPDAT3, LuPDAT4, LuPDAT5, and LuPDAT6 polynucleotides. Plant species of interest for transformation include, without limitation, oilseeds (for example, the linseed plant, rapeseed or canola, peanut, safflower), flax, hemp, camelina, canola, sunflower, olive, palm, oats, wheat, triticale, barley, corn, and legume plants including soybean and pea. In one embodiment, the plant comprises Arabidopsis thaliana.

In one embodiment, the invention comprises a method for producing TAG with enriched PUFA content in oleaginous yeast comprising the steps of:

a) constructing one or more vectors comprising a PDAT polynucleotide as claimed herein;

b) transforming the one or more vectors into a host cell under conditions sufficient for expression of a PDAT encoded by the polynucleotide; and

c) exposing the host cell to a PUFA substrate, wherein the substrate is converted by the PDAT into the TAG product with enriched PUFA content.

The host cell may be engineered to produce or preferentially produce the PUFA substrate. Alternatively, the PUFA substrate may be provided exogenously to the cell.

The following describes specific examples of embodiments of the present invention. It will be appreciated by those skilled in the art that the isolated polynucleotide and polypeptides of the LuPDAT1, LuPDAT2, LuPDAT3, LuPDAT4, LuPDAT5, and LuPDAT6 genes from Linum usitatissimum have industrial and nutritional applications. The LuPDAT1, LuPDAT2, LuPDAT3, LuPDAT4, LuPDAT5, and LuPDAT6 genes encode LuPDAT1, LuPDAT2, LuPDAT3, LuPDAT4, LuPDAT5, and LuPDAT6, respectively. The PDAT polynucleotides and polypeptides may be used in the industrial production of PUFAs using recombinant technology using transformed bacterial, yeast or fungal cells. Transformed cells may be engineered to accumulate TAG which may be incorporated into human food and animal feed applications to produce health supplements or to improve the nutritional quality of products. These examples demonstrate how these genes can be used to produce TAG.

During seed development in flax (Linum usitatissimum), TAGs may be synthesized through a combination of DGAT and PDAT activities. DGAT catalyzes the acyl-CoA-dependent synthesis of TAG, whereas PDAT catalyzes the transfer of a fatty acyl chain from nitrogenous phospholipid to sn-1, 2-diacylglycerol to generate TAG.

To identify flax PDAT genes, a BLAST search (Altschul et al., 1990) was conducted to find the homologous sequences contained in the flax genome database by using A. thaliana PDAT1 as the protein query. The flax genome was shown to contain six PDAT genes. Expression profile analysis indicated that four PDATs (LuPDAT1, LuPDAT3, LuPDAT5 and LuPDAT6) are preferentially expressed in developing flax seed embryos (data not shown).

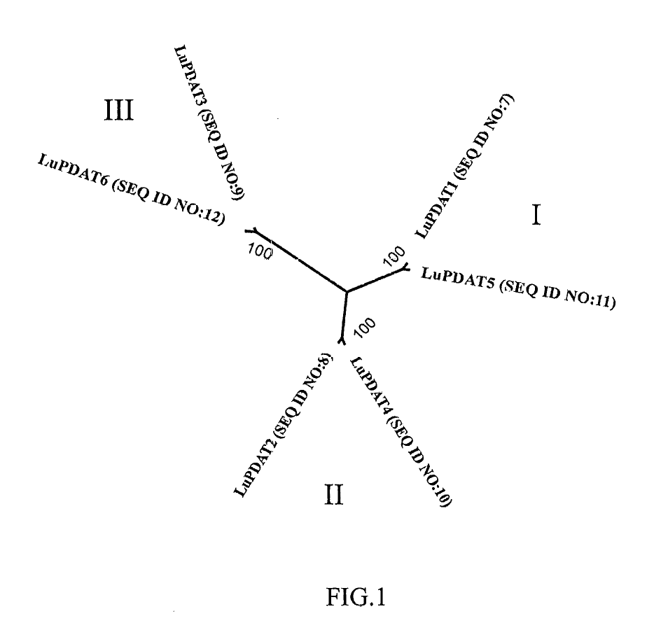

As shown in FIG. 1, phylogenetic analysis indicated that PDATs are divided into three families, each containing two genes as follows: family I (LuPDAT1, LuPDAT5), family II (LuPDAT2, LuPDAT4) and family III (LuPDAT3, LuPDAT6). The identification of LuPDAT gene pairs is consistent with the findings that suggest whole-genome duplication in flax. Because the genes within the duplicated gene pairs share a high degree of sequence identity, their functions are expected be similar.

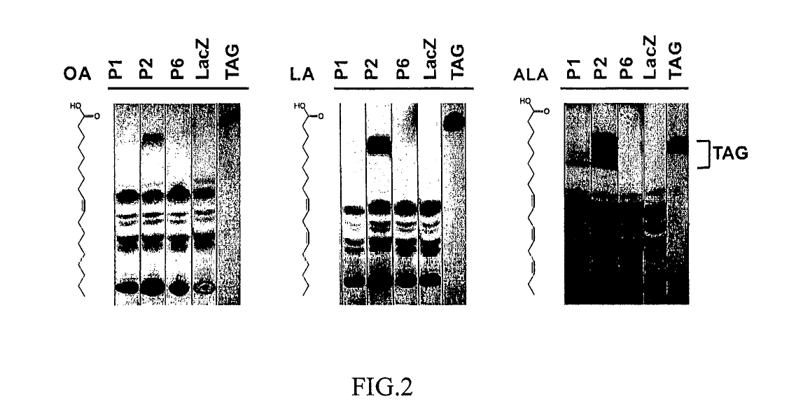

The corresponding cDNAs of LuPDAT1, LuPDAT2, and LuPDAT6 (SEQ ID NOS: 1, 2 and 6 respectively) were expressed in a neutral lipid-deficient yeast quadruple knock-out strain S. cerevisiae (strain H1246) in the presence of specific fatty acids to induce the production of TAG which is enriched with such fatty acids. When yeast was cultured in the presence of ALA, higher amounts of TAGs were produced in yeast expressing LuPDAT1 or LuPDAT2, as may be seen in FIG. 2. However, without being bound by any theory, it appears that the most commonly found fatty acids in yeast including stearic, oleic, palmitic, and palmitoleic acid, are inefficient substrates for LuPDAT1 and LuPDAT2. In addition, LuPDAT1 was not able to produce a visible TAG band on TLC plates when OA or LA was exogenously provided.

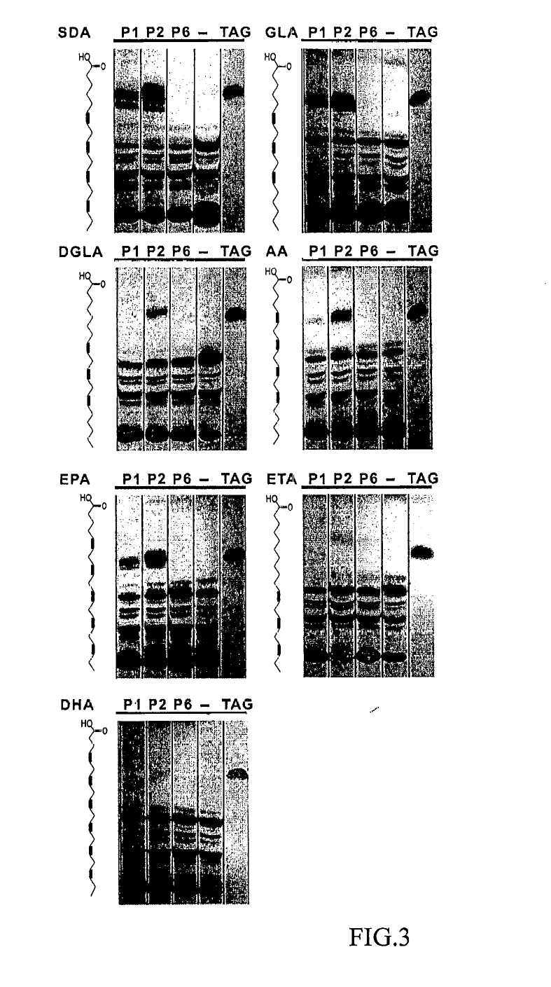

As shown in FIG. 3, LuPDAT1 and LuPDAT2 can also be stimulated by culturing yeast in the presence of other PUFAs, including GLA, SDA, or EPA. The presence of TAG containing only PUFAs in its structure is indicated by the presence of multiple bands in the TAG region. This is evident for LuPDAT1 and LuPDAT2 when yeast cells are supplemented with GLA, SDA, and EPA.

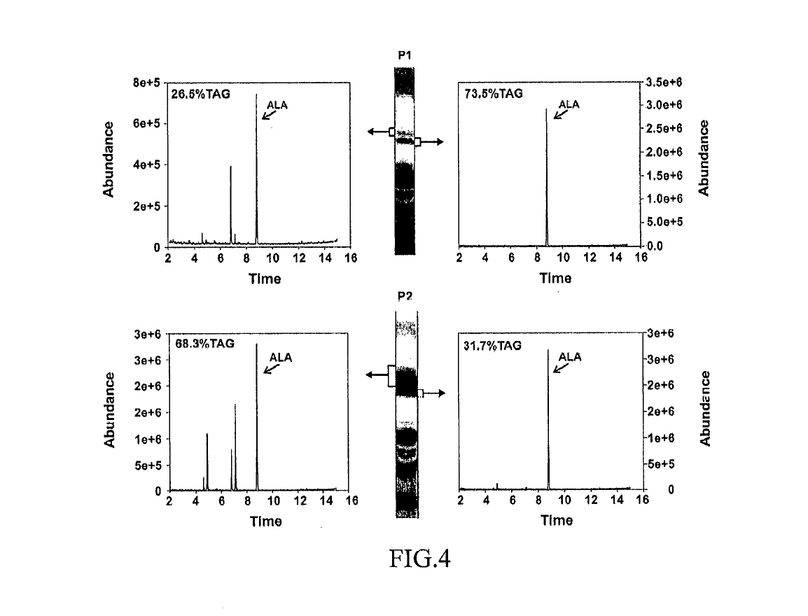

As shown in FIG. 4, LuPDAT1 and LuPDAT2 are capable of synthesizing trilinolenin (an omega-3 polyunsaturated fat) upon culturing yeast in the presence of ALA. The lipid corresponding to the main bands corresponding to TAG (marked in the insert) was scraped from the TLC plate, transmethylated and analyzed through GC-MS. The chromatogram indicates that the lower band contains a single fatty acid that is ALA.

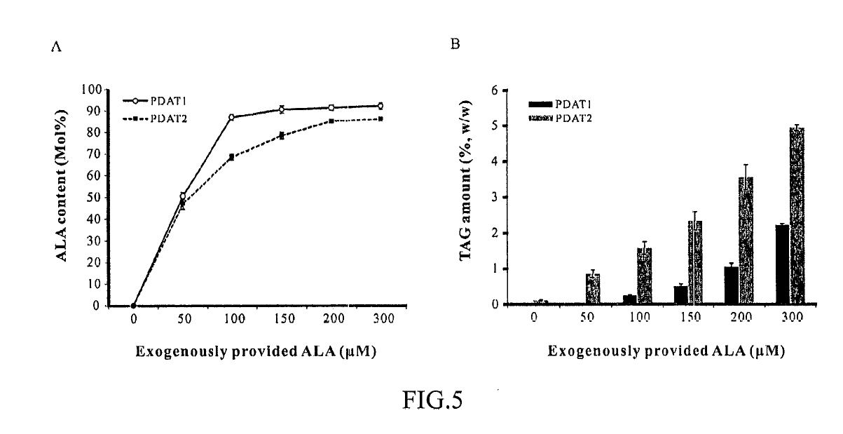

As shown in FIG. 5, both LuPDAT1 and LuPDAT2 have the ability to produce TAG with up to 90% of ALA in yeast H1246. Furthermore, when the concentration of the exogenously provided ALA increased from 0 to 300 μM, the total TAG amount on a dry weight basis increased approximately 168-fold and 44-fold in yeast expressing LuPDAT1 and LuPDAT2, respectively.

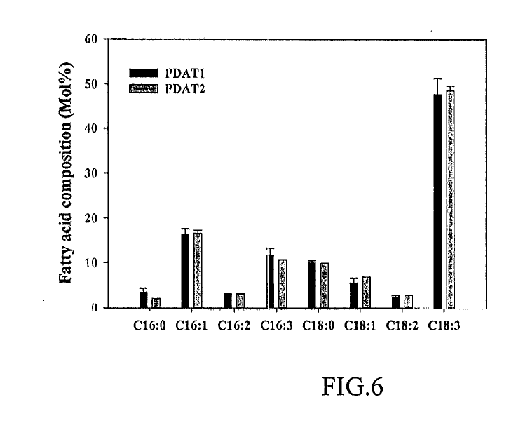

To simulate a natural production of ALA in yeast, LuPDAT1 and LuPDAT2 were individually co-expressed with LuFAD2-1 and LuFAD3B. FIG. 6 indicated that yeast cells expressing LuPDAT1 and LuPDAT2 produced TAG predominantly with ALA.

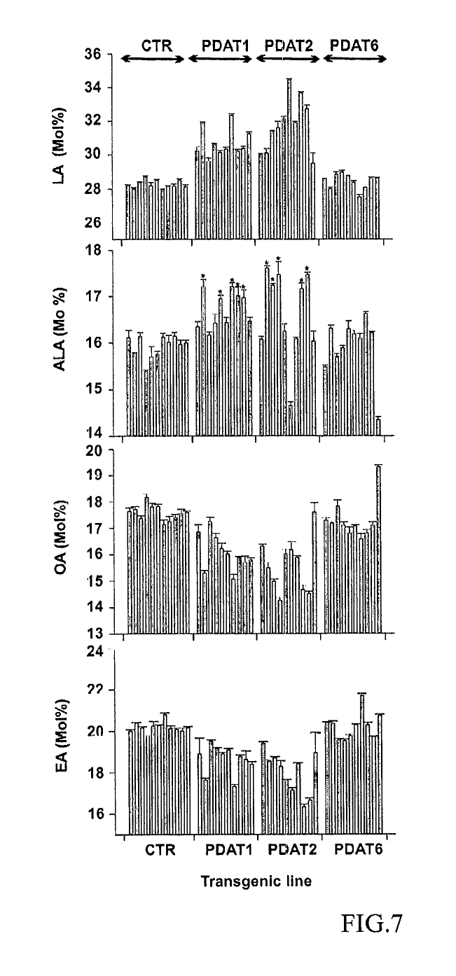

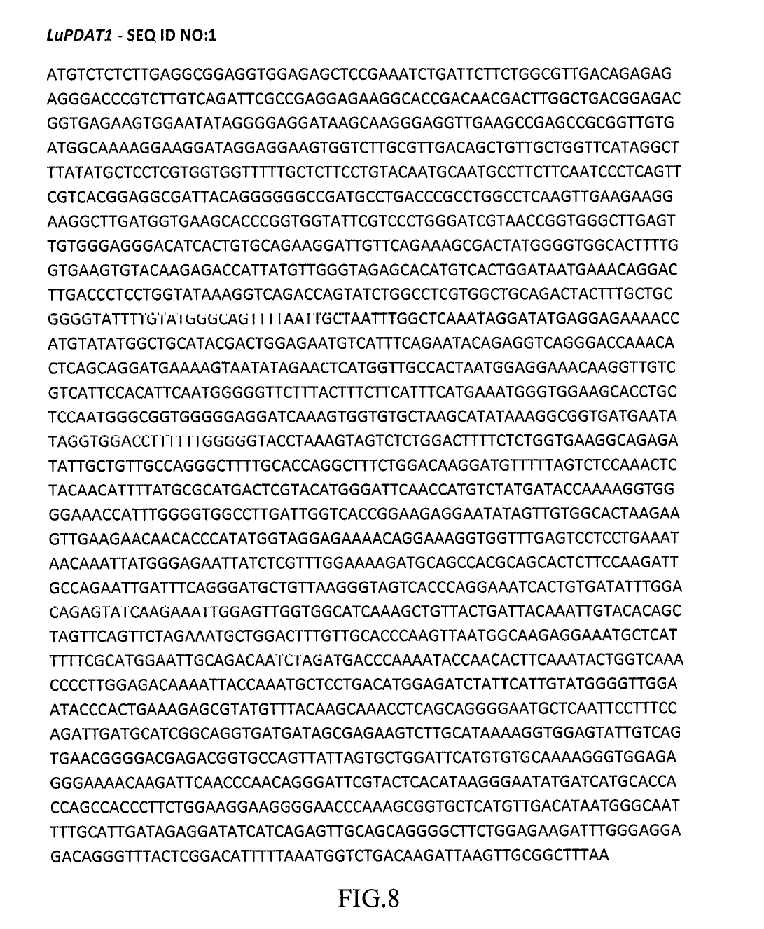

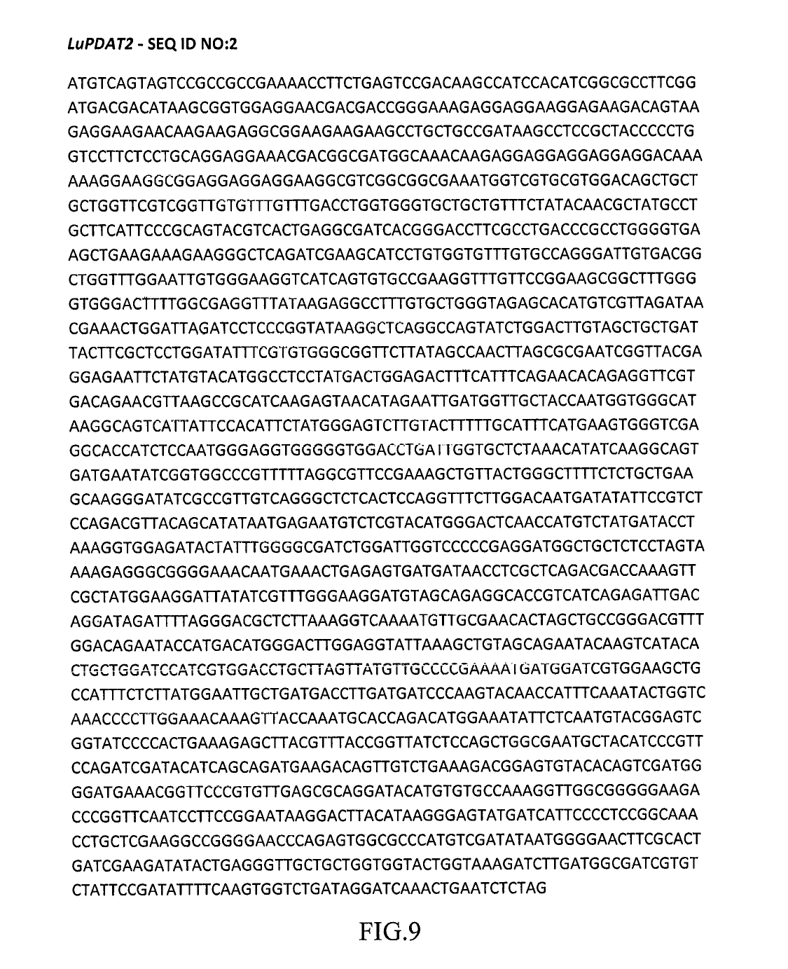

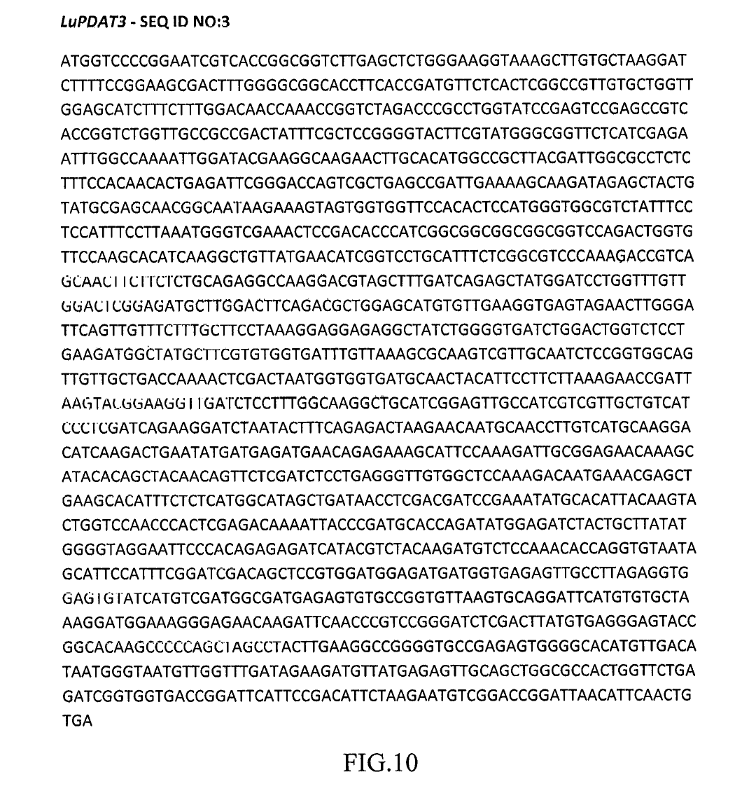

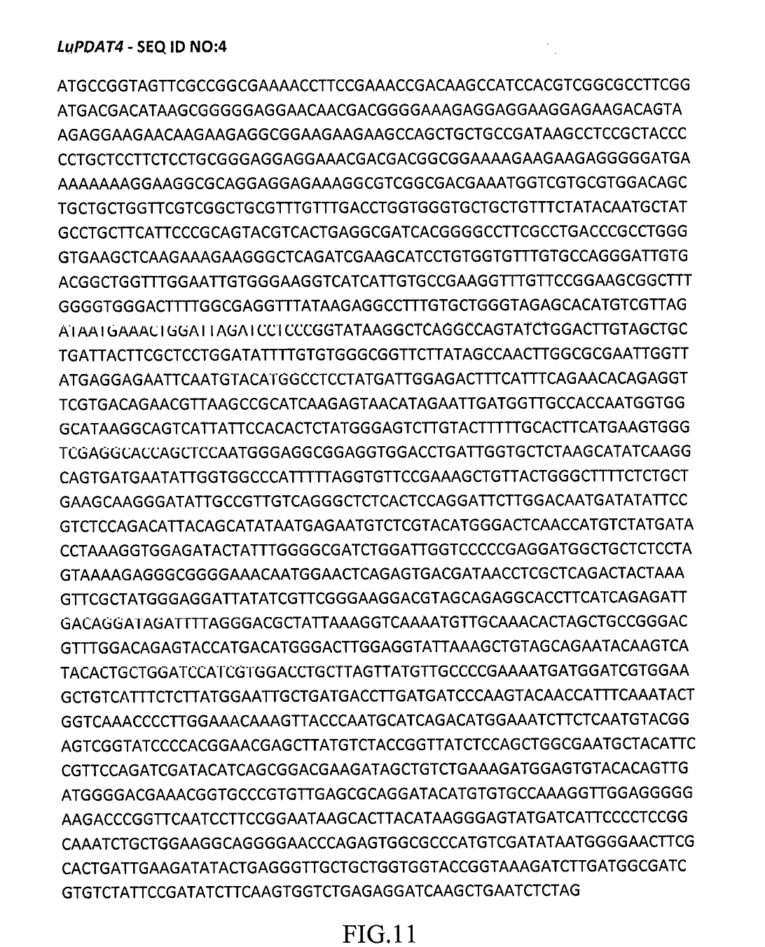

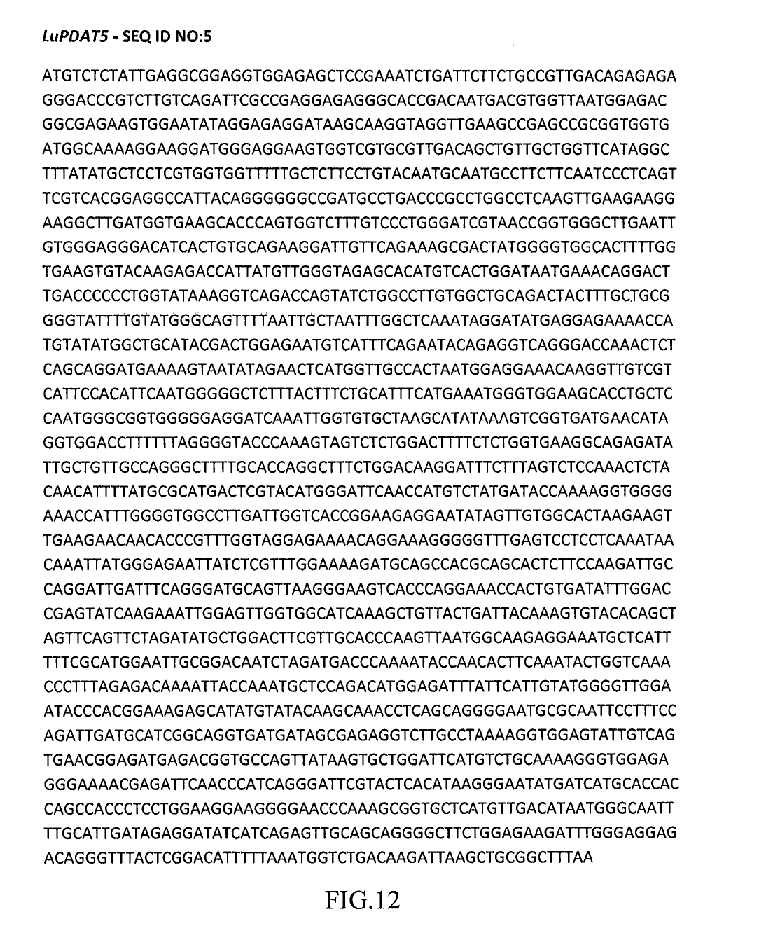

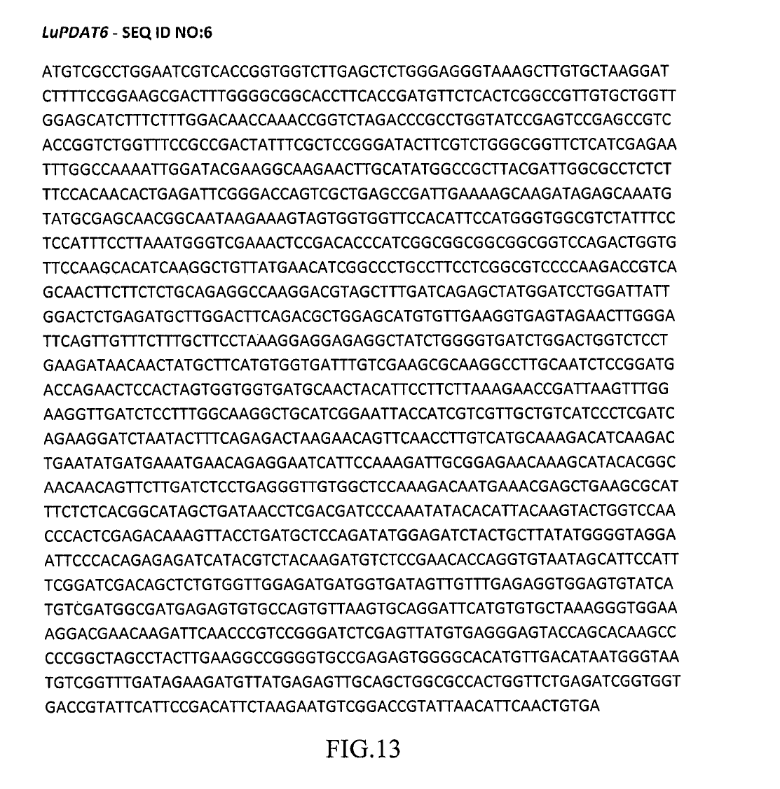

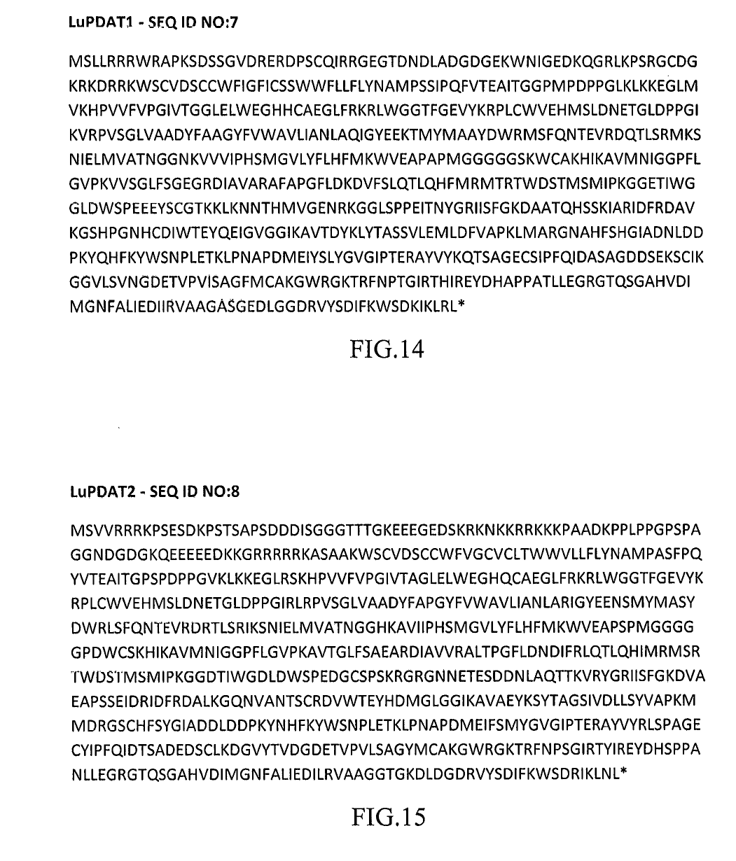

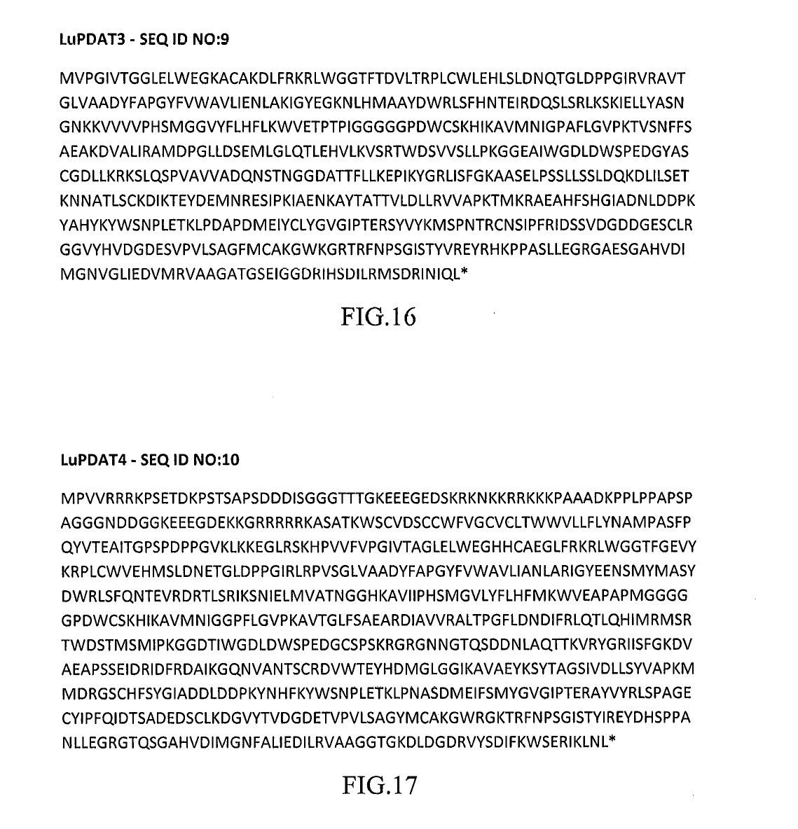

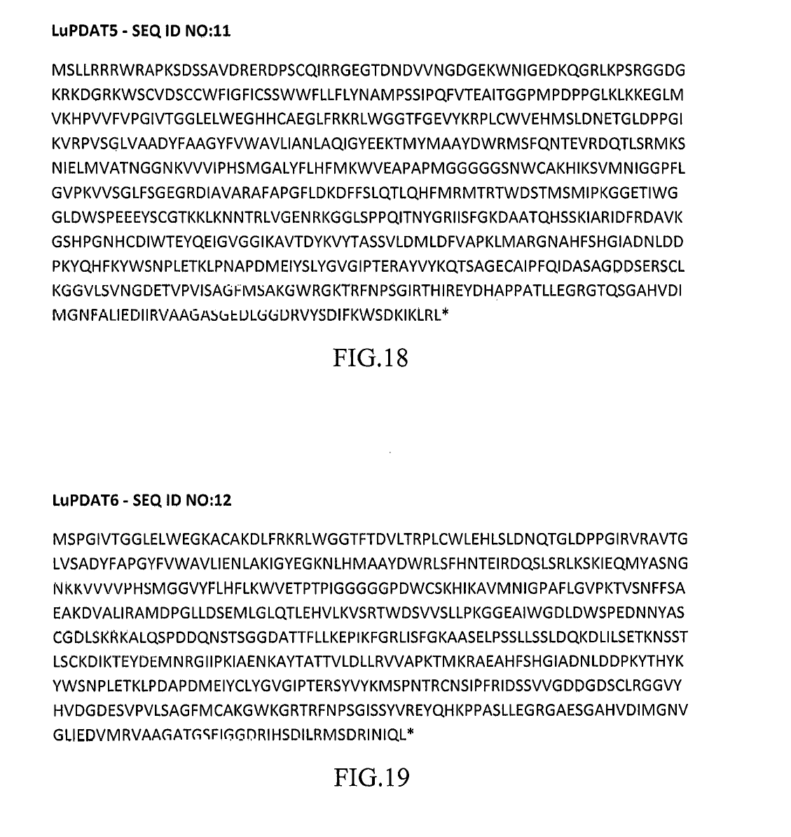

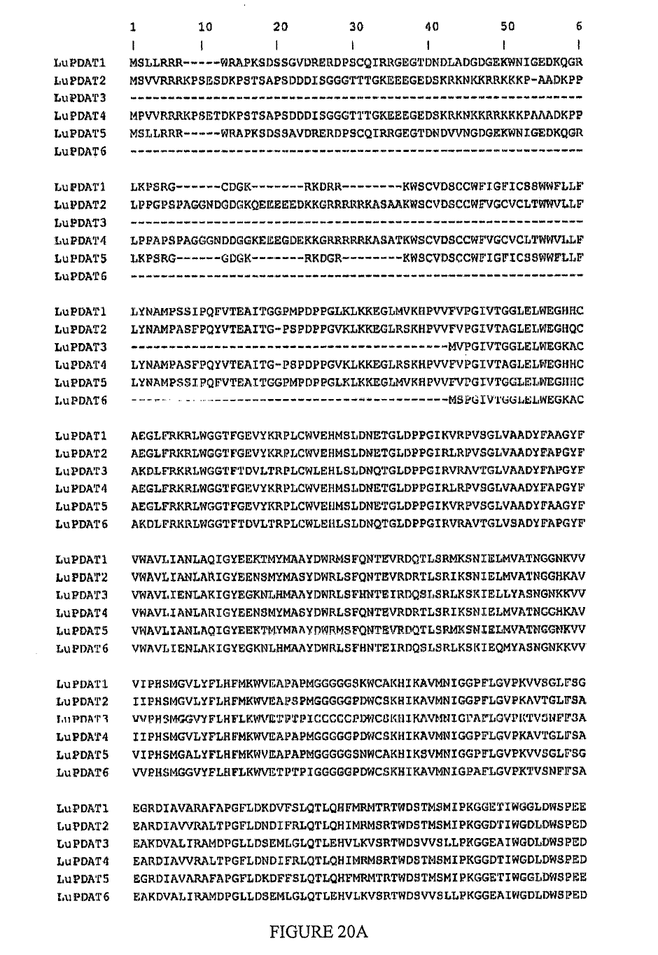

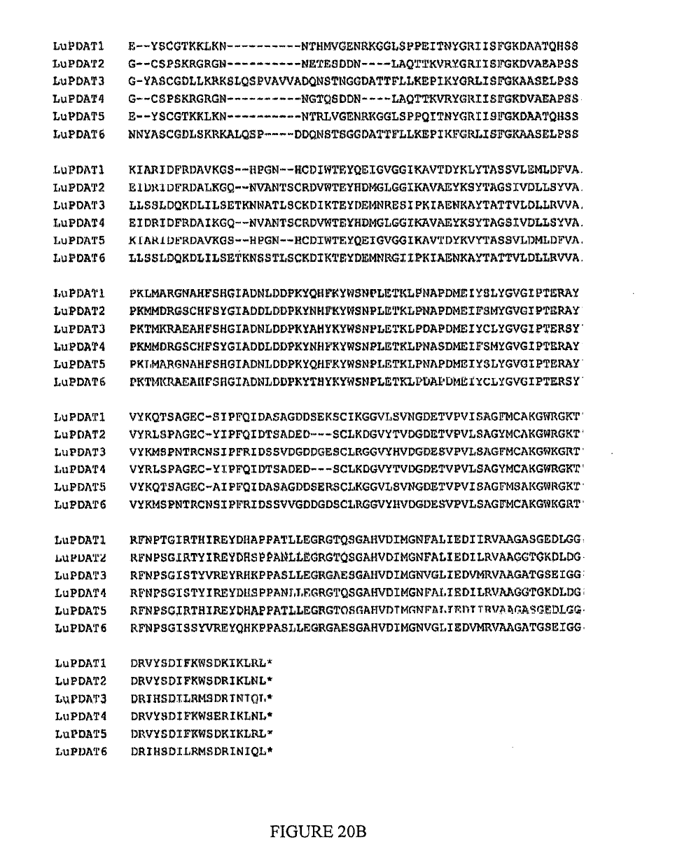

To investigate the functionality of flax PDATs in plant oil biosynthesis, the ORFs of LuPDAT1, LuPDAT2 and LuPDAT6 were expressed in Arabidopsis wild-type Columia under the regulation of the seed-specific napin promoter. As shown in FIG. 7, compared with the empty vector-transformed control wild-type plants, overexpression of the LuPDAT1 and LuPDAT2 significantly increased the PUFA (LA and ALA) content, at the expense of mostly OA and EA. This altered fatty acid composition was not found in Arabidopsis lines overexpressing LuPDAT6.

These results suggest that LuPDAT1 and LuPDAT2 are TAG-synthesizing enzymes, which exhibit preferences for substrates containing not only ALA, but also other PUFAs including GLA, SDA, and EPA. Because the genes within the gene pair share high degree of sequence identity (97% for LuPDAT1/LuPDAT5 and 95.6% for LuPDAT2/LuPDAT4), LuPDAT4 and LuPDAT5 are expected to have similar selectivity. The PDATs with this unique property therefore may be used for production of TAG with enriched PUFA content.

PUFAs are produced in the bound form of TAG, incorporated into all three positions (sn-1, sn-2 and sn-3) of TAG. The substrate selectivity of LuPDAT1 and LuPDAT2 allows the production of TAG comprising a single type of PUFA with at least three, four or five double bonds. The synthesis of TAG containing ALA, GLA, SDA or EPA may thus be produced. Provided that different molecular TAG species are not separated, minor traces of other fatty acids (about 5-10% by weight of TAG) may be present in the end products.

TAG containing two types of PUFAs may also be produced. Depending on the types of PUFAs used for feeding, the end products of TAG may contain a mixture of different omega-3 PUFAs. For example, if both SDA and EPA are used for feeding, the end products of TAG may contain a mixture of SDA and EPA. If both PUFAs (SDA and EPA) are incorporated simultaneously into the TAG by PDAT, the ratio of the two PUFAs may be controlled by the amount of flee fatty acid used for feeding.

Exemplary embodiments of the present invention are described in the following Examples, which are set forth to aid in the understanding of the invention, and should not be construed to limit in any way the scope of the invention as defined in the claims which follow thereafter.

### Example 1

### Identification and Isolation of Flax PDAT Polynucleotides

To identify flax PDAT genes, a BLAST search (Altschul et al., 1990) was conducted to find the homologous sequences contained in the flax genome database (http://www.phytozome.net/flax) by using A. thaliana AtPDAT1 (At5g13640) as the protein query. Six homologous genes, LuPDAT1 (Lus10021564), LuPDAT2 (Lus10037657), LuPDAT3 (Lus10019519), LuPDAT4 (Lus10015639), LuPDAT5 (Lus10017165), and LuPDAT6 (Lus10043360) were identified. The code number in the bracket indicates the gene ID number from the flax genome database. The theoretical molecular weight and isoelectric point values were calculated using the algorithm provided in http://web.expasy.org/compute_pi/.

To isolate the target genes, total RNA was isolated from the embryo of flax (Linum usitatissimm L. cvs. AC Emerson) 12 days after flowering using Plant RNA reagent (Invitrogen, CA). First strand cDNA was synthesized from DNase-treated total RNA using Oligo dT™ primer (Invitrogen) and SuperScript™ III reverse transcriptase (Invitrogen) with oligodT as primer. The target LuPDAT1, LuPDAT2 and LuPDAT6 genes were amplified from the resulting cDNA as the template for 30 cycles of PCR amplification using Platinum Tag DNA Polymerase High Fidelity™ (Invitrogen) with the oligonucleotides set out in Table 1:

TABLE 1
Oligonucleotides for PCR

| | |
|---|---|
| LuPDAT1 | forward 5′-ATGTCTCTCTTGAGGCGGAGGTGG-3′ |
| | (SEQ ID NO: 13) |
| | reverse 5′-TTAAAGCCGCAACTTAATCTTGTC-3′ |
| | (SEQ ID NO: 14) |
| LuPDAT2 | forward 5-ATATGGTACCTACACAATGTCAGTAGTCC |
| | GCCGCCGAAAACCTTC-3′ (SFQ ID NO: 15) |
| | reverse 5′-ATATAAGCTTCTAGAGATTCAGTTTGAT |
| | CCTATCAGACC-3′ (SEQ ID NO: 16) |
| LuPDAT6 | forward 5′-ATATGGTACCTACACAATGTCCCCAGGA |
| | ATCGTCACCGGCGGTC-3′ (SEQ ID NO: 17) |
| | reverse 5′-ATATCTCGAGTCACAGTTGAATGTTAAT |
| | ACGGTCCGAC-3′ (SEQ ID NO: 18) |

PCR was performed under the following temperature cycle program: 95° C. for 2 min; 30 cycles of denaturation (95° C., 20 s), annealing (55° C., 15 s), and extension (68° C., 2.5 min); and a final extension at 68° C. for 2 min. To amplify LuPDAT2 and LuPDAT6, the forward primers introduced a specific restriction site (underlined) and a Kozak translation initiation sequence (italic) to improve the translation of the protein. The ATG start codon is shown in bold and the second amino acid was changed to serine. A specific restriction site was introduced in the reverse primer and the recognition sites are underlined. The PCR products were cloned into the pYES vector collinear to the GAL1 promoter inducible by galactose. The pYES is a modified pYES2.1/V5-HIS vector (Invitrogen, CA), which contains more restriction sites in its multiple cloning site (MCS). For LuPDAT1 which could not be amplified by these specially designed primers, the internal primers were used for amplification. The PCR products were cloned into the pYES2.1/V5-HIS vector using pYES2.1 TOPO™ kit (Invitrogen).

### Example 2

### Expression of LuPDAT Genes in S. cerevisiae Strain H1246 and Production of TAG Rich in PUFAs

The three construct plasmids (pYESLuPDAT1, pYESLuPDAT2 and pYESLuPDAT6) were transformed into S. cerevisiae strain H1246 (Sandager et al. 2002) by using the lithium acetate/SS carrier DNA/PEG method (Gietz and Schiestl, 2007) and transformants were selected on minimal medium plates lacking uracil. Recombinant yeast strains were cultivated in liquid minimal medium containing 2% [w/v] raffinose at 30° C. on a rotary shaker overnight and then induced in minimal medium containing 2% [w/v] galactose and 1% [w/v] raffinose. Yeast transformed with pYESlacZ was used as a negative control.

For the feeding experiments, free fatty acids including OA, LA, ALA, AA, SDA, GLA, DGLA, ETA, EPA and DHA were dissolved at 0.5 M in ethanol. Before yeast induction, the fatty acid solutions were mixed with 0.06% [v/v] tyloxapol, a non-ionic surfactant which dispensed fatty acids evenly in the medium. Yeast was induced in minimal medium containing 2% [w/v] galactose, 1% [w/v] raffinose and 100 μM of each fatty acid.

Induced yeast cultures at the stationary growth stage were harvested, washed and resuspended in 1 mL of 0.9% [w/v] of sodium chloride. Glass beads (0.5 mm) and 2 mL of methanol were added and cells were disrupted by vigorously vortexing for 2 min. Lipids were extracted by adding 4 mL of chloroform. This mixture was vortexed and centrifuged (2000 g) for 2 min. The chloroform phase (lower phase) was collected and the remaining lipids were re-extracted twice by adding 4 mL of chloroform. The collected lipid samples were dried under nitrogen and resuspended in 30 μL of chloroform. The extracted lipids were resolved on the TLC (SIL G25, 0.25 mm, Macherey-Nagel, Germany) with the solvent system hexane/diethyl ether/acetic acid (80:20:1). The developed plate was visualized with 3% cupric acetate [w/v] and 8% phosphoric acid [v/v] followed by charring at 280° C. for 20 min.

Fatty acid analysis was conducted using GC-MS as described previously (Mietkiewska et al. 2011). Briefly, the extracted lipids were developed on the TLC plates by using the same solvent system, but were visualized under UV after spraying with 0.05% primuline. The bands corresponding to triacylglycerol were scraped out and transmethylated with 5% [w/v] sodium methoxide (NaOMe) at room temperature for 30 min. The resulting fatty acid methyl esters (FAMEs) were extracted twice by 2 mL of hexane. The hexane phases were pooled together and dried under a stream of nitrogen and immediately resuspended in 250 μL of iso-octane with 0.1 mg/mL 21:0 standard. FAMEs were analyzed by gas chromatography on an Agilent 6890N GC equipped with DB-23 capillary column (30 m×0.25 mm×0.25 μm) and a 5975 inert XL Mass Selective Detector. The following temperature program was applied: 165° C. hold for 4 min, 10° C. min⁻¹ to 180° C., hold 5 min and 10° C. min⁻¹ to 230° C. hold 5 min.

### Example 3

### Metabolic Engineering Yeast to Enhance ALA Production

To construct the co-expression vectors, the LuFAD2-1 (Krasowska et al, 2007) and LuFAD3B (Vrinten et al. 2005) genes were first amplified using FUR with appropriate primers that allowed to add specific restriction sites (underlined, Table2) to the ends of amplified products and then inserted into MCS 1 and MSC2 of the pESC-URA expression vector (Agilent Technologies, CA), yielding LuFAD2-1-FAD3B/pESC plasmid. The ADH1 terminator: LuFAD2-1:ProGAL10:ProGAL1:LuFAD3B:CYC1 terminator expression cassette of LuFAD2-1-FAD3B/pESC was then excised and subcloned into the recombinant pYES plastids containing LuPDAT1, LuPDAT2 or LuDGAT1 through one-step, isothermal assembly method described by Gibson (2011). The resulting plasmids were referred to as LuFAD2-1-FAD3B-PDAT1/pYES, LuFAD2-1-FAD3B-PDAT2/pYES and LuFAD2-1-FAD3B-DGAT1-1/pYES.

Yeast cells transformed with the resulting plastid were inoculated in the same induction medium for 3 days at 20° C. before cell harvest. Yeast cells transformed with LuFAD2-1-FAD3B/pYES were used as a control.

TABLE 2
Primers used for cloning LuFAD2-FAD3 cassette from pESC
into recombinant pYES vector

| | |
|---|---|
| LuFAD2-1 | forward 5′-ATAGGATCCACCATGGGTGCTGGTGGAAGAAT-3′ |
| | (SEQ ID NO: 19) |
| | reverse 5′-TATGGTACCTCACAGCTTGTTGTTGTACCA-3′ |
| | (SEQ ID NO: 20) |
| LuFAD3B | forward 5′- |
| | CCGGAATTCTACACAATGTCAATGAGCCCTCCAAACTCAATG-3′ |
| | (SEQ ID NO: 21) |
| | reverse 5′-TATGAGCTCTCAGCTGGATTTGGACTTGG-3′ |
| | (SEQ ID NO: 22) |
| LuFAD2- | forward 5′- |
| FAD3 | GAGAGGCGGTTTGCGTATTGGGCGCGCTGAATTGGAGCGACCTCAT |
| | GC-3′ (SEQ ID NO: 23) |
| | reverse 5′- |
| | GTCAGTGAGCGAGGAAGCGGAAGACTGGATCTTCGAGCGTCCCAA |
| | AACC-3′ (SEQ ID NO 24) |
| pYES | forward 5′- |
| | GGTTTTGGGACGCTCGAAGATCCAGTCTTCCGCTTCCTCGCTCACTG |
| | AC-3′ (SEQ ID NO: 25) |
| | reverse 5′- |
| | GCATGAGGTCGCTCCAATTCAGCGCGCCCAATACGCAAACCGCCTC |
| | TC-3′ (SEQ ID NO: 26) |

### Example 4

### Expression of PDAT Polynucleotides in A. thaliana

Agrobacterium tumefaciens strain GV3101 and pGreen/pSoup based dual binary vectors (Hellens et al., 2000) were used for A. thaliana transformation. The coding regions of LuPDAT1, LuPDAT2, and LuPDAT6 were amplified using pYESLuPDAT1, pYESLuPDAT2 and pYESLuPDAT6 plasmids as template with the primers set out in Table 3:

TABLE 3
Primers

| | |
|---|---|
| LuPDAT1 | forward 5′-TATAAAGCTTTACACAATGTCACTCTTGAG |
| | GCGGAGGTGG-3′ (SEQ ID NO: 27) |
| | reverse 5′-TATAGGATCCTTAAAGCCGCAACTTAATCT |
| | TGTCAG-3′ (SEQ ID NO: 28) |
| LuPDAT2 | forward 5′- |
| | ATATCTCGAGTACACAATGTCGGTAGTCCGCCGCCGAAAAC |
| | C-3′ (SEQ ID NO: 29) |
| | reverse 5′-TATATCTAGACTAGAGATTCAGCTTGATCC |
| | TATCAGACC-3′ (SEQ ID NO: 30) |
| LuPDAT6 | forward 5′-TATACCCGGGTAAACAATGTCGCCTGGAAT |
| | CGTCACC-3′ (SEQ ID NO: 31) |
| | reverse 5′-TATACCCGGGTCACAGTTGAATGTTAATAC |
| | GGTCCG-3′ (SEQ ID NO: 32) |

The PCR products were excised by specific restriction enzyme and ligation into the corresponding sites of the pGreen vector under the control of the seed specific napin promoter. The inserts of the constructs were sequenced to confirm their integrity. The resulting construct and the helper plasmid pSoup were co-transformed into Agrobacterium GV3101 by electroporation (Weigel and Glazebrook 2002).

Agrobacterium strains containing the pGreen/pSoup dual binary vectors were used to transform the Arabidopsis wild-type (Columbia) by the floral dipping method (Weigel and Glazebrook 2002). Plants transformed with an empty vector pGreen were used as controls. T1 seeds of transgenic plants were selected on half-strength Murashige and Skoog (MS) agar plates supplemented with 80 μM herbicide phosphinothricin. Transformants were then transferred to soil and grown to maturity to produce T2 seeds. The presence of the target genes was confirmed by gene-specific PCR analysis using DNA extracted from T2 young leaf tissue as template. T2 seeds were collected and used for total lipid and fatty acid analysis.

Total lipid content and the fatty acid composition of T2 seeds were determined by GC-MS. Approximately 10 mg of seeds were placed in a glass tube with triheptadecanoin (C17:0 TAG) were used as a TAG internal standard. Seeds were treated with 2 ml of 3N methanolic-HCL and heated at 80° C. for 16 h. After cooling in ice for 5 min, the FAMES were extracted twice with 2 ml of hexane. The hexane phases were combined and dried under nitrogen. The extracted FAMEs were suspended in 1.5 mL of iso-octane with 0.1 mg/ml 21:0 methyl ester standard and analyzed by GC-MS using the same column and temperature gradient. Total lipid content was determined by multiplying the peak-area ratio of the total fatty acid and the internal standard (C 17:0 TAG) by the initial internal standard amount.

### REFERENCES

The following references and any reference referred to within this specification are are incorporated herein by reference (where permitted) as if reproduced in their entirety. All references are indicative of the level of skill of those skilled in the art to which this invention pertains.
Abeywardena, M. Y., & Patten, G. S. (2011). Role of ω3 longchain polyunsaturated fatty acids in reducing cardio-metabolic risk factors. Endocrine, Metabolic and Immune Disorders—Drug Targets, 11(3), 232-246.
Abbadi, A., Domergue, F., Bauer, J., Napier, J. A., Welti, R., Zähringer, U., Cirpus, P., & Heinz, E. (2004). Biosynthesis of very-long-chain polyunsaturated fatty acids in transgenic oilseeds: Constraints on their accumulation. Plant Cell, 16, 2734-2746.
Altschul, S. F., Gish, W., Miller, W., Myers, E. W., & Lipman, D. J. (1990). Basic local alignment search tool. Journal of Molecular Biology, 215(3), 403-410.
Ausubel, F. M., Bent, R., Kingtone, R. E., Moore, D. J., Smith, J. A., Silverman, J. G., et al. (2000). Current Protocols in Molecular Biology. New York.
Bevan, M., Barker, R., Goldsbrough, A., Jarvis, M., Kavanagh, T., & Iturriaga, G. (1986). The structure and transcription start site of major potato tuber protine gene. Nucleic Acids Research, 14(11), 4625-4638.
Block, M. D., Botterman, J., Vandewiele, M., Dockx, J., Thoen, C., Gossele, V., et al. (1987). Engineering herbicide resistance in plants by expression of a detoxifying enzyme. The EMBO Journal, 6(9), 2513-2518.
Breen, J. P., & Crouch, M. L. (1992). Molecular analysis of a cruciferin storage protein gene family of brassica napus. Plant Molecular Biology, 19(6), 1049-1055,
Browse, J., Grau, L. P., Kinney, A. J., Pierce, Jr., J. W., Wierzbicki, A. M., Yadav, N. S. Fatty acid desaturase genes from plants. U.S. Pat. No. 5,952,544, issued Sep. 14, 1999.
Browse, J. A., Wallis, J. G. & Watts, J. L. Desaturases and methods of using them for synthesis of polyunsaturated fatty acids. U.S. Pat. No. 7,402,735, issued Jul. 22, 2008.
Browse, J. A., Shockey, J. M. & Burgal, J. J. Enhancement of hydroxy fatty acid accumulation in oilseed plants. U.S. Pat. No. 8,101,818, issued Jan. 24, 2012.
Calder, P. C. (2008a). Polyunsaturated fatty acids, inflammatory processes and inflammatory bowel diseases. Molecular Nutrition and Food Research, 52(8), 885-897.
Calder, P. C. (2008b). Session 3: Joint nutrition society and irish nutrition and dietetic institute symposium on ‘nutrition and autoimmune disease’ PUFA, inflammatory processes and rheumatoid arthritis. The Proceedings of the Nutrition Society, 67(4), 409-418.
Chen, T. Process for producing poly-unsaturated fatty acids by oleaginous yeasts. U.S. Pat. No. 7,364,883, issued Apr. 29, 2008.
Cirpus, P., Renz, A., Lerchl, J. & Kuijpers, A. Method for producing multiple unsaturated fatty acids in plants. U.S. Pat. No. 7,893,320, issued Feb. 22, 2011.
Dahlqvist, A., Stahl, U., Lenman, M., Banas, A., Lee, M., Sandager, L., et al. (2000). Phospholipid:Diacylglycerol acyltransferase: An enzyme that catalyzes the acyl-CoA-independent formation of triacylglycerol in yeast and plants. Proceedings of the National Academy of Sciences of the United States of America, 97(12), pp. 6487-6492.
Dahlqvist A., Stahl, U., Lenman, M., Banas, A., Ronne, H. & Stymne, S. Process for the production of triacylglycerols. U.S. Pat. No. 7,635,582, issued Dec. 22, 2009.
Das, U. N. (2006). Essential fatty acids—A review. Current Pharmaceutical Biotechnology, 7, 467-482.
Damude, H. G., Pollak, D. M., Xue, Z., & Zhu, Q. Q. DELTA-5 desaturase and its use in making polyunsaturated fatty acids. U.S. Pat. No. 7,695,950, issued Apr. 13, 2010.
Erikson, O., Hertzberg, M., & Nasholm, T. (2004). A conditional marker gene allowing both positive and negative selection in plants. Nature Biotechnology, 22(4), 455-458.
Gietz, R. D., & Woods, R. A. (2001). Genetic transformation of yeast. BioTechniques, 30(4), 816-20, 822-6, 828 passim.
Gietz, R. D., & Schiestl, R. H. (2007). High-efficiency yeast transformation using the LiAc/SS carrier DNA/PEG method. Nature Protocols, 2, 31-34.
Gunnarsson, N. K., Forster, J. & Nielsen, J. B. Metabolically engineered Saccharomyces cells for the production of polyunsaturated fatty acids. U.S. Pat. No. 7,736,884, issued Jun. 15, 2010.
Harris, W. S., Lemke, S. L., Hansen, S. N., Goldstein, D. A., DiRienzo, M. A., Su, H., et al. (2008). Stearidonic acid-enriched soybean oil increased the omega-3 index, an emerging cardiovascular risk marker, Lipids, 43(9), 805-811,
Hellens, R. P., Anne Edwards, E., Leyland, N. R., Bean, S., & Mullineaux, P. M. (2000). pGreen: A versatile and flexible binary ti vector for agrobacterium-mediated plant transformation. Plant Molecular Biology, 42(6), 819-832.
James, M. J., Ursin, V. M., & Cleland, L. G. (2003). Metabolism of stearidonic acid in human subjects: Comparison with the metabolism of other n-3 fatty acids. The American Journal of Clinical Nutrition, 77(5), 1140-1145.
Kim, H. U., Lee, K. R., Go, Y. S., Jung, J. H., Suh, M. C., & Kim, J. B. (2011). Endoplasmic reticulum-located PDAT1-2 from castor bean enhances hydroxy fatty acid accumulation in transgenic plants. Plant & Cell Physiology, 52(6), 983-993.
Knutzon, D., Mukerji, P., Huang, Y., Thurmond, J. & Chaudhary, S. Methods and compositions for synthesis of long chain poly-unsaturated fatty acids. U.S. Pat. No. 5,968,809, issued Oct. 19, 1999.
Krasowska, A., Dziadkowiec, D., Polinceusz, A., Plonka, A., & Lukaszewicz, M. (2007). Cloning of flax oleic fatty acid desaturase and its expression in yeast. Journal of the American Oil Chemists' Society, 84, 809-816.
Lerchl, J., Renz, A., Heinz, E., Domergue, F., & Zahringer, U. Production of polyunsaturated fatty acids, novel biosynthesis genes, and novel plant expression constructs. U.S. Pat. No. 8,088,974, issued Jan. 3, 2012.
Martin, C. E. & Mitchell, A. Synthetic fatty acid desaturase gene for expression in plants. U.S. Pat. No. 6,825,335, issued Nov. 30, 2004.
Mietkiewska, E., Siloto, R. M. P., Dewald, J., Shah, S., Brindley, D. N., & Weselake, R. J. (2011) Lipins from plants are phosphatidate phosphatases that restore lipid synthesis in a pah1δ mutant strain of Saccharomyces cerevisiae. FEBS Journal, 278, 764-775.
Mukerji, P., Pereira, S. L., & Huang, Y. Desaturase genes, enzymes encoded thereby, and uses thereof. U.S. Pat. No. 7,723,503, issued May 25, 2010.
Napier, J. A. & Sayanova, O. DELTA 6-desaturases from Primulaceae, expressing plants and PUFA-containing oils. U.S. Pat. No. 7,554,008, issued Jun. 30, 2009,
Okamuro, J. K., & Goldberg, R. B. (1989). Regulation of plant gene expression: General principles. In M. Marcus (Ed.), The Biochemistry of Plants (2nd edition ed., pp. 4-4-51). San Diego: Academic Press.
Pereira, S. L., Huang, Y. S., Bobik, E. G., Kinney, A. J., Stecca, K. L., Packer, J. C. L., & Mukerji, P. (2004). A novel ω3-fatty acid desaturase involved in the biosynthesis of eicosapentaenoic acid. Biochemical Journal, 378, 665-671.
Qi, B., Fraser, T., Mugford, S., Dobson, G., Sayanova, O., Butler, J., Napier, J. A., Stobart, A. K., & Lazarus, C. M. (2004). Production of very long chain polyunsaturated omega-3 and omega-6 fatty acids in plants. Nature Biotechnology, 22, 739-745.
Qiu, X. & Hong, H. Fad4, Fad5, Fad5-2, and Fad6, novel fatty acid desaturase family members and uses thereof. U.S. Pat. No. 7,671,252, issued Mar. 2, 2010.
Rakoczy-Trojanowska, M. (2002). Alternative methods of plant transformation—a short review. Cellular & Molecular Biology Letters, 7(3), 849-858.
Ruiz-Lopez, N., Sayanova, O., Napier, J. A., & Haslam, R. P. (2012). Metabolic engineering of the omega-3 long chain polyunsaturated fatty acid biosynthetic pathway into transgenic plants. Journal of Experimental Botany, 63(7), 2397-2410.
Sambrook, J., Fritsch, E. F., & Maniatis, T. (1989). Molecular Cloning: A Laboratory Manual (2nd ed.). New York: Cold Spring Harbor Press.
Sandager, L., Gustavsson, M. H., Ståhl, U., Dahlqvist, A., Wiberg, E., Banas, A., et al. (2002). Storage lipid synthesis is non-essential in yeast. Journal of Biological Chemistry, 277(8), 6478-6482,
Saravanan, P., Davidson, N. C., Schmidt, E. B., & Calder, P. C. (2010). Cardiovascular effects of marine omega-3 fatty acids. Lancet, 376(9740), 540-550.
Sinclair, A. J., Attar-Bashi, N. M., and Lib, D. (2003). What is the role of alpha-linolenic acid for mammals?. Lipids, 37, 1113-1123
Thomas, T. L., Reddy, A. S., Nuccio, M., Nunberg, A. N. & Freyssinet, G. L. Production of gamma-linolenic acid by a DELTA 6-desaturase, U.S. Pat. No. 5,614,393, issued Mar. 25, 1997.
Tocher, D. R., Dick, J. R., MacGlaughlin, P., & Bell, J. G. (2006). Effect of diets enriched in Delta6 desaturated fatty acids (18:3n-6 and 18:4n-3), on growth, fatty acid composition and highly unsaturated fatty acid synthesis in two populations of arctic charr (salvelinus alpinus L.). Comparative Biochemistry and Physiology. Part B, Biochemistry & Molecular Biology, 144(2), 245-253.
Vrinten, P., Hu, Z., Munchinsky, M. A., Rowland, G., & Qiu, X. (2005). Two FAD3 desaturase genes control the level of linolenic acid in flax seed. Plant Physiology, 139, 79-87.
Wada, H., Gombos, Z., & Murata, N. (1990). Enhancement of chilling tolerance of a cyanobacterium by genetic manipulation of fatty acid desaturation. Nature, 347(6289), 200-203.
Weigel, D., & Glazebrook, J. (2002). Arabidopsis: A Laboratory Manual, Cold Spring Harbor Laboratory Press, Cold Spring Harbor, N.Y.
Weiss, S. B., Kennedy, E. P., and Kiyasu, J. Y. (1960). The enzymatic synthesis of triglycerides. The Journal of Biological Chemistry, 235, 40-44.
Whelan, J. (2009). Dietary stearidonic acid is a long chain (n-3) polyunsaturated fatty acid with potential health benefits. The Journal of Nutrition, 139(1), 5-10.
Yadav, N. S. & Zhang, H. DELTA 15 desaturases suitable for altering levels of polyunsaturated fatty acids in oleaginous plants and yeast. U.S. Pat. No. 7,659,120, issued Feb. 9, 2010.
Yoon, K., Han, D., Li, Y., Sommerfeld, M., & Hu, Q. (2006). Phospholipid:diacylglycerol acyltransferase is a multifunctional enzyme involved in membrane lipid turnover and degradation while synthesizing triacylglycerol in the unicellular green microalga Chlamydomonas reinhardtii. Plant Cell, 24, 3708-3724.

1. An isolated polynucleotide sequence encoding a protein or polypeptide comprising or consisting of an amino acid sequence selected from the group consisting of SEQ ID NOS: 7, 8, 9, 10, 11, or 12, respective biologically active variants and biologically active portions thereof, with respective sequences having at least 80% identity based on the Clustal W method of alignment, and wherein the variants or portions have phospholipid: diacylglycerol acyltransferase (PDAT) activity.

2. The isolated polynucleotide of claim 1, wherein the polynucleotide encodes a polypeptide having PDAT activity and comprising the amino acid sequence of SEQ ID NO: 7, or an amino acid sequence having PDAT activity and having at least 80% sequence identity therewith.

3. The isolated polynucleotide of claim 1, wherein the polynucleotide encodes a polypeptide having PDAT activity and comprising the amino acid sequence of SEQ ID NO: 8, or an amino acid sequence having PDAT activity and having at least 80% sequence identity therewith.

4. The isolated polynucleotide of claim 1, wherein the polynucleotide encodes a polypeptide having PDAT activity and comprising the amino acid sequence of SEQ ID NO: 9, or an amino acid sequence having PDAT activity and having at least 80% sequence identity therewith.

5. The isolated polynucleotide of claim 1, wherein the polynucleotide encodes a polypeptide having PDAT activity and comprising the amino acid sequence of SEQ ID NO: 10, or an amino acid sequence having PDAT activity and having at least 80% sequence identity therewith.

6. The isolated polynucleotide of claim 1, wherein the polynucleotide encodes a polypeptide having PDAT activity and comprising the amino acid sequence of SEQ ID NO: 11, or an amino acid sequence having PDAT activity and having at least 80% sequence identity therewith.

7. The isolated polynucleotide of claim 1, wherein the polynucleotide encodes a polypeptide having PDAT activity and comprising the amino acid sequence of SEQ ID NO: 12, or an amino acid sequence having PDAT activity and having at least 80% sequence identity therewith.

8. The isolated polynucleotide of claim 1, wherein the polynucleotide comprises the nucleotide sequence of SEQ ID NO: 1, 2, 3, 4, 5 or 6.

9. The isolated polynucleotide of claim 1, wherein the encoded polypeptide comprises an amino acid sequence having at least 85%, 90%, at least 95%, at least 96%, at least 97%, at least 98%, or at least 99% sequence identity to one of SEQ ID NO: 7, 8, 9, 10, 11 or 12.

10. A recombinant expression vector comprising a polynucleotide sequence of claim 1 operably linked with transcriptional and translational regulatory regions or sequences to provide for expression of the at least one polynucleotide sequence in a host cell.

11. A microbial cell comprising the recombinant expression vector of claim 10.

12. The microbial cell of claim 11, wherein the cell comprises Saccharomyces cerevisiae engineered to be deficient in TAG synthesis.

13. The microbial cell of claim 12 which co-expresses a recombinant elongase or desaturase enzyme, or both.

14. (canceled)

15. A transgenic plant, plant cell, plant seed, callus, plant embryo, microspore-derived embryo, or microspore, comprising the recombinant expression vector of claim 10.

16. (canceled)

17. A method for producing TAG with enriched polyunsaturated fatty acid content comprising the steps of:
a) transforming a recombinant expression vector of claim 10 into a host cell under conditions sufficient for expression of a PDAT; and
b) exposing the host cell to a fatty acid substrate, wherein the substrate is converted by the PDAT into the TAG product with enriched polyunsaturated fatty acid.

18. The method of claim 17, wherein the fatty acid substrate comprises one or more of γ-linolenic acid, α-linolenic acid, stearidonic acid or eicosapentaenoic acid.

19. The method of claim 18 wherein the fatty acid substrate is provided exogenously, or the host cell is engineered to produce or preferentially produce the fatty acid substrate.

20. The method of claim 18, wherein the polypeptide comprises the amino acid sequence selected from the group consisting of SEQ ID NO: 7, 8, 10, and 11, and sequences having at least 80% identity thereto, and the fatty acid substrate comprises one or more of α-linolenic acid, γ-linolenic acid, stearidonic acid, or eicosapentaenoic acid.

21. The method of claim 17 wherein the host cell is a yeast cell.

22. The method of claim 17 wherein the host cell is a plant cell.

23. The method of claim 22 wherein the host cell is comprised in a linseed, rapeseed, canola, peanut, safflower, flax, hemp, camelina, soybean, pea, sunflower, olive, palm, oats, wheat, triticale, barley, corn, and legume plant, plant cell, plant seed, callus, plant embryo, or microspore-derived embryo or microspore.

24. The method of claim 21 wherein the yeast cell is engineered to be deficient in TAG synthesis.

25. The method of claim 24 wherein the yeast cell co-expresses a recombinant elongase or desaturase enzyme.


SEQUENCE LISTING (32 sequences)

SEQ ID NO: 1 - DNA, 2088 bp, Linum usitatissimum
atgtctctcttgaggcggaggtggagagctccgaaatctgattcttctggcgttgacagagagagggacccgtcttgtcagattcgccgaggagaaggcaccgacaacgacttggctgacggagacggtgagaagtggaatataggggaggataagcaagggaggttgaagccgagccgcggttgtgatggcaaaaggaaggataggaggaagtggtcttgcgttgacagctgttgctggttcataggctttatatgctcctcgtggtggtttttgctcttcctgtacaatgcaatgccttcttcaatccctcagttcgtcacggaggcgattacaggggggccgatgcctgacccgcctggcctcaagttgaagaaggaaggcttgatggtgaagcacccggtggtattcgtccctgggatcgtaaccggtgggcttgagttgtgggagggacatcactgtgcagaaggattgttcagaaagcgactatggggtggcacttttggtgaagtgtacaagagaccattatgttgggtagagcacatgtcactggataatgaaacaggacttgaccctcctggtataaaggtcagaccagtatctggcctcgtggctgcagactactttgctgcggggtattttgtatgggcagttttaattgctaatttggctcaaataggatatgaggagaaaaccatgtatatggctgcatacgactggagaatgtcatttcagaatacagaggtcagggaccaaacactcagcaggatgaaaagtaatatagaactcatggttgccactaatggaggaaacaaggttgtcgtcattccacattcaatgggggttctttactttcttcatttcatgaaatgggtggaagcacctgctccaatgggcggtgggggaggatcaaagtggtgtgctaagcatataaaggcggtgatgaatataggtggaccttttttgggggtacctaaagtagtctctggacttttctctggtgaaggcagagatattgctgttgccagggcttttgcaccaggctttctggacaaggatgtttttagtctccaaactctacaacattttatgcgcatgactcgtacatgggattcaaccatgtctatgataccaaaaggtggggaaaccatttggggtggccttgattggtcaccggaagaggaatatagttgtggcactaagaagttgaagaacaacacccatatggtaggagaaaacaggaaaggtggtttgagtcctcctgaaataacaaattatgggagaattatctcgtttggaaaagatgcagccacgcagcactcttccaagattgccagaattgatttcagggatgctgttaagggtagtcacccaggaaatcactgtgatatttggacagagtatcaagaaattggagttggtggcatcaaagctgttactgattacaaattgtacacagctagttcagttctagaaatgctggactttgttgcacccaagttaatggcaagaggaaatgctcatttttcgcatggaattgcagacaatctagatgacccaaaataccaacacttcaaatactggtcaaaccccttggagacaaaattaccaaatgctcctgacatggagatctattcattgtatggggttggaatacccactgaaagagcgtatgtttacaagcaaacctcagcaggggaatgctcaattcctttccagattgatgcatcggcaggtgatgatagcgagaagtcttgcataaaaggtggagtattgtcagtgaacggggacgagacggtgccagttattagtgctggattcatgtgtgcaaaagggtggagagggaaaacaagattcaacccaacagggattcgtactcacataagggaatatgatcatgcaccaccagccacccttctggaaggaaggggaacccaaagcggtgctcatgttgacataatgggcaattttgcattgatagaggatatcatcagagttgcagcaggggcttctggagaagatttgggaggagacagggtttactcggacatttttaaatggtctgacaagattaagttgcggctttaa

SEQ ID NO: 2 - DNA, 2145 bp, Linum usitatissimum
atgtcagtagtccgccgccgaaaaccttctgagtccgacaagccatccacatcggcgccttcggatgacgacataagcggtggaggaacgacgaccgggaaagaggaggaaggagaagacagtaagaggaagaacaagaagaggcggaagaagaagcctgctgccgataagcctccgctaccccctggtccttctcctgcaggaggaaacgacggcgatggcaaacaagaggaggaggaggaggacaaaaaaggaaggcggaggaggaggaaggcgtcggcggcgaaatggtcgtgcgtggacagctgctgctggttcgtcggttgtgtttgtttgacctggtgggtgctgctgtttctatacaacgctatgcctgcttcattcccgcagtacgtcactgaggcgatcacgggaccttcgcctgacccgcctggggtgaagctgaagaaagaagggctcagatcgaagcatcctgtggtgtttgtgccagggattgtgacggctggtttggaattgtgggaaggtcatcagtgtgccgaaggtttgttccggaagcggctttggggtgggacttttggcgaggtttataagaggcctttgtgctgggtagagcacatgtcgttagataacgaaactggattagatcctcccggtataaggctcaggccagtatctggacttgtagctgctgattacttcgctcctggatatttcgtgtgggcggttcttatagccaacttagcgcgaatcggttacgaggagaattctatgtacatggcctcctatgactggagactttcatttcagaacacagaggttcgtgacagaacgttaagccgcatcaagagtaacatagaattgatggttgctaccaatggtgggcataaggcagtcattattccacattctatgggagtcttgtactttttgcatttcatgaagtgggtcgaggcaccatctccaatgggaggtgggggtggacctgattggtgctctaaacatatcaaggcagtgatgaatatcggtggcccgtttttaggcgttccgaaagctgttactgggcttttctctgctgaagcaagggatatcgccgttgtcagggctctcactccaggtttcttggacaatgatatattccgtctccagacgttacagcatataatgagaatgtctcgtacatgggactcaaccatgtctatgatacctaaaggtggagatactatttggggcgatctggattggtcccccgaggatggctgctctcctagtaaaagagggcggggaaacaatgaaactgagagtgatgataacctcgctcagacgaccaaagttcgctatggaaggattatatcgtttgggaaggatgtagcagaggcaccgtcatcagagattgacaggatagattttagggacgctcttaaaggtcaaaatgttgcgaacactagctgccgggacgtttggacagaataccatgacatgggacttggaggtattaaagctgtagcagaatacaagtcatacactgctggatccatcgtggacctgcttagttatgttgccccgaaaatgatggatcgtggaagctgccatttctcttatggaattgctgatgaccttgatgatcccaagtacaaccatttcaaatactggtcaaaccccttggaaacaaagttaccaaatgcaccagacatggaaatattctcaatgtacggagtcggtatccccactgaaagagcttacgtttaccggttatctccagctggcgaatgctacatcccgttccagatcgatacatcagcagatgaagacagttgtctgaaagacggagtgtacacagtcgatggggatgaaacggttcccgtgttgagcgcaggatacatgtgtgccaaaggttggcgggggaagacccggttcaatccttccggaataaggacttacataagggagtatgatcattcccctccggcaaacctgctcgaaggccggggaacccagagtggcgcccatgtcgatataatggggaacttcgcactgatcgaagatatactgagggttgctgctggtggtactggtaaagatcttgatggcgatcgtgtctattccgatattttcaagtggtctgataggatcaaactgaatctctag

SEQ ID NO: 3 - DNA, 1728 bp, Linum usitatissimum
atggtccccggaatcgtcaccggcggtcttgagctctgggaaggtaaagcttgtgctaaggatcttttccggaagcgactttggggcggcaccttcaccgatgttctcactcggccgttgtgctggttggagcatctttctttggacaaccaaaccggtctagacccgcctggtatccgagtccgagccgtcaccggtctggttgccgccgactatttcgctccggggtacttcgtatgggcggttctcatcgagaatttggccaaaattggatacgaaggcaagaacttgcacatggccgcttacgattggcgcctctctttccacaacactgagattcgggaccagtcgctgagccgattgaaaagcaagatagagctactgtatgcgagcaacggcaataagaaagtagtggtggttccacactccatgggtggcgtctatttcctccatttccttaaatgggtcgaaactccgacacccatcggcggcggcggcggtccagactggtgttccaagcacatcaaggctgttatgaacatcggtcctgcatttctcggcgtcccaaagaccgtcagcaacttcttctctgcagaggccaaggacgtagctttgatcagagctatggatcctggtttgttggactcggagatgcttggacttcagacgctggagcatgtgttgaaggtgagtagaacttgggattcagttgtttctttgcttcctaaaggaggagaggctatctggggtgatctggactggtctcctgaagatggctatgcttcgtgtggtgatttgttaaagcgcaagtcgttgcaatctccggtggcagttgttgctgaccaaaactcgactaatggtggtgatgcaactacattccttcttaaagaaccgattaagtacggaaggttgatctcctttggcaaggctgcatcggagttgccatcgtcgttgctgtcatccctcgatcagaaggatctaatactttcagagactaagaacaatgcaaccttgtcatgcaaggacatcaagactgaatatgatgagatgaacagagaaagcattccaaagattgcggagaacaaagcatacacagctacaacagttctcgatctcctgagggttgtggctccaaagacaatgaaacgagctgaagcacatttctctcatggcatagctgataacctcgacgatccgaaatatgcacattacaagtactggtccaacccactcgagacaaaattacccgatgcaccagatatggagatctactgcttatatggggtaggaattcccacagagagatcatacgtctacaagatgtctccaaacaccaggtgtaatagcattccatttcggatcgacagctccgtggatggagatgatggtgagagttgccttagaggtggagtgtatcatgtcgatggcgatgagagtgtgccggtgttaagtgcaggattcatgtgtgctaaaggatggaaagggagaacaagattcaacccgtccgggatctcgacttatgtgagggagtaccggcacaagcccccagctagcctacttgaaggccggggtgccgagagtggggcacatgttgacataatgggtaatgttggtttgatagaagatgttatgagagttgcagctggcgccactggttctgagatcggtggtgaccggattcattccgacattctaagaatgtcggaccggattaacattcaactgtga

SEQ ID NO: 4 - DNA, 2148 bp, Linum usitatissimum
atgccggtagttcgccggcgaaaaccttccgaaaccgacaagccatccacgtcggcgccttcggatgacgacataagcgggggaggaacaacgacggggaaagaggaggaaggagaagacagtaagaggaagaacaagaagaggcggaagaagaagccagctgctgccgataagcctccgctaccccctgctccttctcctgcgggaggaggaaacgacgacggcggaaaagaagaagagggggatgaaaaaaaaggaaggcgcaggaggagaaaggcgtcggcgacgaaatggtcgtgcgtggacagctgctgctggttcgtcggctgcgtttgtttgacctggtgggtgctgctgtttctatacaatgctatgcctgcttcattcccgcagtacgtcactgaggcgatcacggggccttcgcctgacccgcctggggtgaagctcaagaaagaagggctcagatcgaagcatcctgtggtgtttgtgccagggattgtgacggctggtttggaattgtgggaaggtcatcattgtgccgaaggtttgttccggaagcggctttggggtgggacttttggcgaggtttataagaggcctttgtgctgggtagagcacatgtcgttagataatgaaactggattagatcctcccggtataaggctcaggccagtatctggacttgtagctgctgattacttcgctcctggatattttgtgtgggcggttcttatagccaacttggcgcgaattggttatgaggagaattcaatgtacatggcctcctatgattggagactttcatttcagaacacagaggttcgtgacagaacgttaagccgcatcaagagtaacatagaattgatggttgccaccaatggtgggcataaggcagtcattattccacactctatgggagtcttgtactttttgcacttcatgaagtgggtcgaggcaccagctccaatgggaggcggaggtggacctgattggtgctctaagcatatcaaggcagtgatgaatattggtggcccatttttaggtgttccgaaagctgttactgggcttttctctgctgaagcaagggatattgccgttgtcagggctctcactccaggattcttggacaatgatatattccgtctccagacattacagcatataatgagaatgtctcgtacatgggactcaaccatgtctatgatacctaaaggtggagatactatttggggcgatctggattggtcccccgaggatggctgctctcctagtaaaagagggcggggaaacaatggaactcagagtgacgataacctcgctcagactactaaagttcgctatgggaggattatatcgttcgggaaggacgtagcagaggcaccttcatcagagattgacaggatagattttagggacgctattaaaggtcaaaatgttgcaaacactagctgccgggacgtttggacagagtaccatgacatgggacttggaggtattaaagctgtagcagaatacaagtcatacactgctggatccatcgtggacctgcttagttatgttgccccgaaaatgatggatcgtggaagctgtcatttctcttatggaattgctgatgaccttgatgatcccaagtacaaccatttcaaatactggtcaaaccccttggaaacaaagttacccaatgcatcagacatggaaatcttctcaatgtacggagtcggtatccccacggaacgagcttatgtctaccggttatctccagctggcgaatgctacattccgttccagatcgatacatcagcggacgaagatagctgtctgaaagatggagtgtacacagttgatggggacgaaacggtgcccgtgttgagcgcaggatacatgtgtgccaaaggttggagggggaagacccggttcaatccttccggaataagcacttacataagggagtatgatcattcccctccggcaaatctgctggaaggcaggggaacccagagtggcgcccatgtcgatataatggggaacttcgcactgattgaagatatactgagggttgctgctggtggtaccggtaaagatcttgatggcgatcgtgtctattccgatatcttcaagtggtctgagaggatcaagctgaatctctag

SEQ ID NO: 5 - DNA, 2088 bp, Linum usitatissimum
atgtctctattgaggcggaggtggagagctccgaaatctgattcttctgccgttgacagagagagggacccgtcttgtcagattcgccgaggagagggcaccgacaatgacgtggttaatggagacggcgagaagtggaatataggagaggataagcaaggtaggttgaagccgagccgcggtggtgatggcaaaaggaaggatgggaggaagtggtcgtgcgttgacagctgttgctggttcataggctttatatgctcctcgtggtggtttttgctcttcctgtacaatgcaatgccttcttcaatccctcagttcgtcacggaggccattacaggggggccgatgcctgacccgcctggcctcaagttgaagaaggaaggcttgatggtgaagcacccagtggtctttgtccctgggatcgtaaccggtgggcttgaattgtgggagggacatcactgtgcagaaggattgttcagaaagcgactatggggtggcacttttggtgaagtgtacaagagaccattatgttgggtagagcacatgtcactggataatgaaacaggacttgacccccctggtataaaggtcagaccagtatctggccttgtggctgcagactactttgctgcggggtattttgtatgggcagttttaattgctaatttggctcaaataggatatgaggagaaaaccatgtatatggctgcatacgactggagaatgtcatttcagaatacagaggtcagggaccaaactctcagcaggatgaaaagtaatatagaactcatggttgccactaatggaggaaacaaggttgtcgtcattccacattcaatgggggctctttactttctgcatttcatgaaatgggtggaagcacctgctccaatgggcggtgggggaggatcaaattggtgtgctaagcatataaagtcggtgatgaacataggtggaccttttttaggggtacccaaagtagtctctggacttttctctggtgaaggcagagatattgctgttgccagggcttttgcaccaggctttctggacaaggatttctttagtctccaaactctacaacattttatgcgcatgactcgtacatgggattcaaccatgtctatgataccaaaaggtggggaaaccatttggggtggccttgattggtcaccggaagaggaatatagttgtggcactaagaagttgaagaacaacacccgtttggtaggagaaaacaggaaagggggtttgagtcctcctcaaataacaaattatgggagaattatctcgtttggaaaagatgcagccacgcagcactcttccaagattgccaggattgatttcagggatgcagttaagggaagtcacccaggaaaccactgtgatatttggaccgagtatcaagaaattggagttggtggcatcaaagctgttactgattacaaagtgtacacagctagttcagttctagatatgctggacttcgttgcacccaagttaatggcaagaggaaatgctcatttttcgcatggaattgcggacaatctagatgacccaaaataccaacacttcaaatactggtcaaaccctttagagacaaaattaccaaatgctccagacatggagatttattcattgtatggggttggaatacccacggaaagagcatatgtatacaagcaaacctcagcaggggaatgcgcaattcctttccagattgatgcatcggcaggtgatgatagcgagaggtcttgcctaaaaggtggagtattgtcagtgaacggagatgagacggtgccagttataagtgctggattcatgtctgcaaaagggtggagagggaaaacgagattcaacccatcagggattcgtactcacataagggaatatgatcatgcaccaccagccaccctcctggaaggaaggggaacccaaagcggtgctcatgttgacataatgggcaattttgcattgatagaggatatcatcagagttgcagcaggggcttctggagaagatttgggaggagacagggtttactcggacatttttaaatggtctgacaagattaagctgcggctttaa

SEQ ID NO: 6 - DNA, 1719 bp, Linum usitatissimum
atgtcgcctggaatcgtcaccggtggtcttgagctctgggagggtaaagcttgtgctaaggatcttttccggaagcgactttggggcggcaccttcaccgatgttctcactcggccgttgtgctggttggagcatctttctttggacaaccaaaccggtctagacccgcctggtatccgagtccgagccgtcaccggtctggtttccgccgactatttcgctccgggatacttcgtctgggcggttctcatcgagaatttggccaaaattggatacgaaggcaagaacttgcatatggccgcttacgattggcgcctctctttccacaacactgagattcgggaccagtcgctgagccgattgaaaagcaagatagagcaaatgtatgcgagcaacggcaataagaaagtagtggtggttccacattccatgggtggcgtctatttcctccatttccttaaatgggtcgaaactccgacacccatcggcggcggcggcggtccagactggtgttccaagcacatcaaggctgttatgaacatcggccctgccttcctcggcgtccccaagaccgtcagcaacttcttctctgcagaggccaaggacgtagctttgatcagagctatggatcctggattattggactctgagatgcttggacttcagacgctggagcatgtgttgaaggtgagtagaacttgggattcagttgtttctttgcttcctaaaggaggagaggctatctggggtgatctggactggtctcctgaagataacaactatgcttcatgtggtgatttgtcgaagcgcaaggccttgcaatctccggatgaccagaactccactagtggtggtgatgcaactacattccttcttaaagaaccgattaagtttggaaggttgatctcctttggcaaggctgcatcggaattaccatcgtcgttgctgtcatccctcgatcagaaggatctaatactttcagagactaagaacagttcaaccttgtcatgcaaagacatcaagactgaatatgatgaaatgaacagaggaatcattccaaagattgcggagaacaaagcatacacggcaacaacagttcttgatctcctgagggttgtggctccaaagacaatgaaacgagctgaagcgcatttctctcacggcatagctgataacctcgacgatcccaaatatacacattacaagtactggtccaacccactcgagacaaagttacctgatgctccagatatggagatctactgcttatatggggtaggaattcccacagagagatcatacgtctacaagatgtctccgaacaccaggtgtaatagcattccatttcggatcgacagctctgtggttggagatgatggtgatagttgtttgagaggtggagtgtatcatgtcgatggcgatgagagtgtgccagtgttaagtgcaggattcatgtgtgctaaagggtggaaaggacgaacaagattcaacccgtccgggatctcgagttatgtgagggagtaccagcacaagcccccggctagcctacttgaaggccggggtgccgagagtggggcacatgttgacataatgggtaatgtcggtttgatagaagatgttatgagagttgcagctggcgccactggttctgagatcggtggtgaccgtattcattccgacattctaagaatgtcggaccgtattaacattcaactgtga

SEQ ID NO: 7 - protein, 1964 aa, Linum usitatissimum
METSERLELEARGARGARGTRPARGALAPRLYSSERASPSERSERGLYVALASPARGGLARGASPPRSERCYSGLNILEARGARGGLYGLGLYTHRASPASNASPLEALAASPGLYASPGLYGLLYSTRPASNILEGLYGLASPLYSGLNGLYARGLELYSPRSERARGGLYCYSASPGLYLYSARGLYSASPARGARGLYSTRPSERCYSVALASPSERCYSCYSTRPPHEILEGLYPHEILECYSSERSERTRPTRPPHELELEPHELETYRASNALAMETPRSERSERILEPRGLNPHEVALTHRGLALAILETHRGLYGLYPRMETPRASPPRPRGLYLELYSLELYSLYSGLGLYLEMETVALLYSHISPRVALVALPHEVALPRGLYILEVALTHRGLYGLYLEGLLETRPGLGLYHISHISCYSALAGLGLYLEPHEARGLYSARGLETRPGLYGLYTHRPHEGLYGLVALTYRLYSARGPRLECYSTRPVALGLHISMETSERLEASPASNGLTHRGLYLEASPPRPRGLYILELYSVALARGPRVALSERGLYLEVALALAALAASPTYRPHEALAALAGLYTYRPHEVALTRPALAVALLEILEALAASNLEALAGLNILEGLYTYRGLGLLYSTHRMETTYRMETALAALATYRASPTRPARGMETSERPHEGLNASNTHRGLVALARGASPGLNTHRLESERARGMETLYSSERASNILEGLLEMETVALALATHRASNGLYGLYASNLYSVALVALVALILEPRHISSERMETGLYVALLETYRPHELEHISPHEMETLYSTRPVALGLALAPRALAPRMETGLYGLYGLYGLYGLYSERLYSTRPCYSALALYSHISILELYSALAVALMETASNILEGLYGLYPRPHELEGLYVALPRLYSVALVALSERGLYLEPHESERGLYGLGLYARGASPILEALAVALALAARGALAPHEALAPRGLYPHELEASPLYSASPVALPHESERLEGLNTHRLEGLNHISPHEMETARGMETTHRARGTHRTRPASPSERTHRMETSERMETILEPRLYSGLYGLYGLTHRILETRPGLYGLYLEASPTRPSERPRGLGLGLTYRSERCYSGLYTHRLYSLYSLELYSASNASNTHRHISMETVALGLYGLASNARGLYSGLYGLYLESERPRPRGLILETHRASNTYRGLYARGILEILESERPHEGLYLYSASPALAALATHRGLNHISSERSERLYSILEALAARGILEASPPHEARGASPALAVALLYSGLYSERHISPRGLYASNHISCYSASPILETRPTHRGLTYRGLNGLILEGLYVALGLYGLYILELYSALAVALTHRASPTYRLYSLETYRTHRALASERSERVALLEGLMETLEASPPHEVALALAPRLYSLEMETALAARGGLYASNALAHISPHESERHISGLYILEALAASPASNLEASPASPPRLYSTYRGLNHISPHELYSTYRTRPSERASNPRLEGLTHRLYSLEPRASNALAPRASPMETGLILETYRSERLETYRGLYVALGLYILEPRTHRGLARGALATYRVALTYRLYSGLNTHRSERALAGLYGLCYSSERILEPRPHEGLNILEASPALASERALAGLYASPASPSERGLLYSSERCYSILELYSGLYGLYVALLESERVALASNGLYASPGLTHRVALPRVALILESERALAGLYPHEMETCYSALALYSGLYTRPARGGLYLYSTHRARGPHEASNPRTHRGLYILEARGTHRHISILEARGGLTYRASPHISALAPRPRALATHRLELEGLGLYARGGLYTHRGLNSERGLYALAHISVALASPILEMETGLYASNPHEALALEILEGLASPILEILEARGVALALAALAGLYALASERGLYGLASPLEGLYGLYASPARGVALTYRSERASPILEPHELYSTRPSERASPLYSILELYSLEARGLE

SEQ ID NO: 8 - protein, 2008 aa, Linum usitatissimum
METSERVALVALARGARGARGLYSPRSERGLSERASPLYSPRSERTHRSERALAPRSERASPASPASPILESERGLYGLYGLYTHRTHRTHRGLYLYSGLGLGLGLYGLASPSERLYSARGLYSASNLYSLYSARGARGLYSLYSLYSPRALAALAASPLYSPRPRLEPRPRGLYPRSERPRALAGLYGLYASNASPGLYASPGLYLYSGLNGLGLGLGLGLASPLYSLYSGLYARGARGARGARGARGLYSALASERALAALALYSTRPSERCYSVALASPSERCYSCYSTRPPHEVALGLYCYSVALCYSLETHRTRPTRPVALLELEPHELETYRASNALAMETPRALASERPHEPRGLNTYRVALTHRGLALAILETHRGLYPRSERPRASPPRPRGLYVALLYSLELYSLYSGLGLYLEARGSERLYSHISPRVALVALPHEVALPRGLYILEVALTHRALAGLYLEGLLETRPGLGLYHISGLNCYSALAGLGLYLEPHEARGLYSARGLETRPGLYGLYTHRPHEGLYGLVALTYRLYSARGPRLECYSTRPVALGLHISMETSERLEASPASNGLTHRGLYLEASPPRPRGLYILEARGLEARGPRVALSERGLYLEVALALAALAASPTYRPHEALAPRGLYTYRPHEVALTRPALAVALLEILEALAASNLEALAARGILEGLYTYRGLGLASNSERMETTYRMETALASERTYRASPTRPARGLESERPHEGLNASNTHRGLVALARGASPARGTHRLESERARGILELYSSERASNILEGLLEMETVALALATHRASNGLYGLYHISLYSALAVALILEILEPRHISSERMETGLYVALLETYRPHELEHISPHEMETLYSTRPVALGLALAPRSERPRMETGLYGLYGLYGLYGLYPRASPTRPCYSSERLYSHISILELYSALAVALMETASNILEGLYGLYPRPHELEGLYVALPRLYSALAVALTHRGLYLEPHESERALAGLALAARGASPILEALAVALVALARGALALETHRPRGLYPHELEASPASNASPILEPHEARGLEGLNTHRLEGLNHISILEMETARGMETSERARGTHRTRPASPSERTHRMETSERMETILEPRLYSGLYGLYASPTHRILETRPGLYASPLEASPTRPSERPRGLASPGLYCYSSERPRSERLYSARGGLYARGGLYASNASNGLTHRGLSERASPASPASNLEALAGLNTHRTHRLYSVALARGTYRGLYARGILEILESERPHEGLYLYSASPVALALAGLALAPRSERSERGLILEASPARGILEASPPHEARGASPALALELYSGLYGLNASNVALALAASNTHRSERCYSARGASPVALTRPTHRGLTYRHISASPMETGLYLEGLYGLYILELYSALAVALALAGLTYRLYSSERTYRTHRALAGLYSERILEVALASPLELESERTYRVALALAPRLYSMETMETASPARGGLYSERCYSHISPHESERTYRGLYILEALAASPASPLEASPASPPRLYSTYRASNHISPHELYSTYRTRPSERASNPRLEGLTHRLYSLEPRASNALAPRASPMETGLILEPHESERMETTYRGLYVALGLYILEPRTHRGLARGALATYRVALTYRARGLESERPRALAGLYGLCYSTYRILEPRPHEGLNILEASPTHRSERALAASPGLASPSERCYSLELYSASPGLYVALTYRTHRVALASPGLYASPGLTHRVALPRVALLESERALAGLYTYRMETCYSALALYSGLYTRPARGGLYLYSTHRARGPHEASNPRSERGLYILEARGTHRTYRILEARGGLTYRASPHISSERPRPRALAASNLELEGLGLYARGGLYTHRGLNSERGLYALAHISVALASPILEMETGLYASNPHEALALEILEGLASPILELEARGVALALAALAGLYGLYTHRGLYLYSASPLEASPGLYASPARGVALTYRSERASPILEPHELYSTRPSERASPARGILELYSLEASNLE

SEQ ID NO: 9 - protein, 1605 aa, Linum usitatissimum
METVALPRGLYILEVALTHRGLYGLYLEGLLETRPGLGLYLYSALACYSALALYSASPLEPHEARGLYSARGLETRPGLYGLYTHRPHETHRASPVALLETHRARGPRLECYSTRPLEGLHISLESERLEASPASNGLNTHRGLYLEASPPRPRGLYILEARGVALARGALAVALTHRGLYLEVALALAALAASPTYRPHEALAPRGLYTYRPHEVALTRPALAVALLEILEGLASNLEALALYSILEGLYTYRGLGLYLYSASNLEHISMETALAALATYRASPTRPARGLESERPHEHISASNTHRGLILEARGASPGLNSERLESERARGLELYSSERLYSILEGLLELETYRALASERASNGLYASNLYSLYSVALVALVALVALPRHISSERMETGLYGLYVALTYRPHELEHISPHELELYSTRPVALGLTHRPRTHRPRILEGLYGLYGLYGLYGLYPRASPTRPCYSSERLYSHISILELYSALAVALMETASNILEGLYPRALAPHELEGLYVALPRLYSTHRVALSERASNPHEPHESERALAGLALALYSASPVALALALEILEARGALAMETASPPRGLYLELEASPSERGLMETLEGLYLEGLNTHRLEGLHISVALLELYSVALSERARGTHRTRPASPSERVALVALSERLELEPRLYSGLYGLYGLALAILETRPGLYASPLEASPTRPSERPRGLASPGLYTYRALASERCYSGLYASPLELELYSARGLYSSERLEGLNSERPRVALALAVALVALALAASPGLNASNSERTHRASNGLYGLYASPALATHRTHRPHELELELYSGLPRILELYSTYRGLYARGLEILESERPHEGLYLYSALAALASERGLLEPRSERSERLELESERSERLEASPGLNLYSASPLEILELESERGLTHRLYSASNASNALATHRLESERCYSLYSASPILELYSTHRGLTYRASPGLMETASNARGGLSERILEPRLYSILEALAGLASNLYSALATYRTHRALATHRTHRVALLEASPLELEARGVALVALALAPRLYSTHRMETLYSARGALAGLALAHISPHESERHISGLYILEALAASPASNLEASPASPPRLYSTYRALAHISTYRLYSTYRTRPSERASNPRLEGLTHRLYSLEPRASPALAPRASPMETGLILETYRCYSLETYRGLYVALGLYILEPRTHRGLARGSERTYRVALTYRLYSMETSERPRASNTHRARGCYSASNSERILEPRPHEARGILEASPSERSERVALASPGLYASPASPGLYGLSERCYSLEARGGLYGLYVALTYRHISVALASPGLYASPGLSERVALPRVALLESERALAGLYPHEMETCYSALALYSGLYTRPLYSGLYARGTHRARGPHEASNPRSERGLYILESERTHRTYRVALARGGLTYRARGHISLYSPRPRALASERLELEGLGLYARGGLYALAGLSERGLYALAHISVALASPILEMETGLYASNVALGLYLEILEGLASPVALMETARGVALALAALAGLYALATHRGLYSERGLILEGLYGLYASPARGILEHISSERASPILELEARGMETSERASPARGILEASNILEGLNLE

SEQ ID NO: 10 - protein, 2014 aa, Linum usitatissimum
METPRVALVALARGARGARGLYSPRSERGLTHRASPLYSPRSERTHRSERALAPRSERASPASPASPILESERGLYGLYGLYTHRTHRTHRGLYLYSGLGLGLGLYGLASPSERLYSARGLYSASNLYSLYSARGARGLYSLYSLYSPRALAALAALAASPLYSPRPRLEPRPRALAPRSERPRALAGLYGLYGLYASNASPASPGLYGLYLYSGLGLGLGLYASPGLLYSLYSGLYARGARGARGARGARGLYSALASERALATHRLYSTRPSERCYSVALASPSERCYSCYSTRPPHEVALGLYCYSVALCYSLETHRTRPTRPVALLELEPHELETYRASNALAMETPRALASERPHEPRGLNTYRVALTHRGLALAILETHRGLYPRSERPRASPPRPRGLYVALLYSLELYSLYSGLGLYLEARGSERLYSHISPRVALVALPHEVALPRGLYILEVALTHRALAGLYLEGLLETRPGLGLYHISHISCYSALAGLGLYLEPHEARGLYSARGLETRPGLYGLYTHRPHEGLYGLVALTYRLYSARGPRLECYSTRPVALGLHISMETSERLEASPASNGLTHRGLYLEASPPRPRGLYILEARGLEARGPRVALSERGLYLEVALALAALAASPTYRPHEALAPRGLYTYRPHEVALTRPALAVALLEILEALAASNLEALAARGILEGLYTYRGLGLASNSERMETTYRMETALASERTYRASPTRPARGLESERPHEGLNASNTHRGLVALARGASPARGTHRLESERARGILELYSSERASNILEGLLEMETVALALATHRASNGLYGLYHISLYSALAVALILEILEPRHISSERMETGLYVALLETYRPHELEHISPHEMETLYSTRPVALGLALAPRALAPRMETGLYGLYGLYGLYGLYPRASPTRPCYSSERLYSHISILELYSALAVALMETASNILEGLYGLYPRPHELEGLYVALPRLYSALAVALTHRGLYLEPHESERALAGLALAARGASPILEALAVALVALARGALALETHRPRGLYPHELEASPASNASPILEPHEARGLEGLNTHRLEGLNHISILEMETARGMETSERARGTHRTRPASPSERTHRMETSERMETILEPRLYSGLYGLYASPTHRILETRPGLYASPLEASPTRPSERPRGLASPGLYCYSSERPRSERLYSARGGLYARGGLYASNASNGLYTHRGLNSERASPASPASNLEALAGLNTHRTHRLYSVALARGTYRGLYARGILEILESERPHEGLYLYSASPVALALAGLALAPRSERSERGLILEASPARGILEASPPHEARGASPALAILELYSGLYGLNASNVALALAASNTHRSERCYSARGASPVALTRPTHRGLTYRHISASPMETGLYLEGLYGLYILELYSALAVALALAGLTYRLYSSERTYRTHRALAGLYSERILEVALASPLELESERTYRVALALAPRLYSMETMETASPARGGLYSERCYSHISPHESERTYRGLYILEALAASPASPLEASPASPPRLYSTYRASNHISPHELYSTYRTRPSERASNPRLEGLTHRLYSLEPRASNALASERASPMETGLILEPHESERMETTYRGLYVALGLYILEPRTHRGLARGALATYRVALTYRARGLESERPRALAGLYGLCYSTYRILEPRPHEGLNILEASPTHRSERALAASPGLASPSERCYSLELYSASPGLYVALTYRTHRVALASPGLYASPGLTHRVALPRVALLESERALAGLYTYRMETCYSALALYSGLYTRPARGGLYLYSTHRARGPHEASNPRSERGLYILESERTHRTYRILEARGGLTYRASPHISSERPRPRALAASNLELEGLGLYARGGLYTHRGLNSERGLYALAHISVALASPILEMETGLYASNPHEALALEILEGLASPILELEARGVALALAALAGLYGLYTHRGLYLYSASPLEASPGLYASPARGVALTYRSERASPILEPHELYSTRPSERGLARGILELYSLEASNLE

SEQ ID NO: 11 - protein, 1966 aa, Linum usitatissimum
METSERLELEARGARGARGTRPARGALAPRLYSSERASPSERSERALAVALASPARGGLARGASPPRSERCYSGLNILEARGARGGLYGLGLYTHRASPASNASPVALVALASNGLYASPGLYGLLYSTRPASNILEGLYGLASPLYSGLNGLYARGLELYSPRSERARGGLYGLYASPGLYLYSARGLYSASPGLYARGLYSTRPSERCYSVALASPSERCYSCYSTRPPHEILEGLYPHEILECYSSERSERTRPTRPPHELELEPHELETYRASNALAMETPRSERSERILEPRGLNPHEVALTHRGLALAILETHRGLYGLYPRMETPRASPPRPRGLYLELYSLELYSLYSGLGLYLEMETVALLYSHISPRVALVALPHEVALPRGLYILEVALTHRGLYGLYLEGLLETRPGLGLYHISHISCYSALAGLGLYLEPHEARGLYSARGLETRPGLYGLYTHRPHEGLYGLVALTYRLYSARGPRLECYSTRPVALGLHISMETSERLEASPASNGLTHRGLYLEASPPRPRGLYILELYSVALARGPRVALSERGLYLEVALALAALAASPTYRPHEALAALAGLYTYRPHEVALTRPALAVALLEILEALAASNLEALAGLNILEGLYTYRGLGLLYSTHRMETTYRMETALAALATYRASPTRPARGMETSERPHEGLNASNTHRGLVALARGASPGLNTHRLESERARGMETLYSSERASNILEGLLEMETVALALATHRASNGLYGLYASNLYSVALVALVALILEPRHISSERMETGLYALALETYRPHELEHISPHEMETLYSTRPVALGLALAPRALAPRMETGLYGLYGLYGLYGLYSERASNTRPCYSALALYSHISILELYSSERVALMETASNILEGLYGLYPRPHELEGLYVALPRLYSVALVALSERGLYLEPHESERGLYGLGLYARGASPILEALAVALALAARGALAPHEALAPRGLYPHELEASPLYSASPPHEPHESERLEGLNTHRLEGLNHISPHEMETARGMETTHRARGTHRTRPASPSERTHRMETSERMETILEPRLYSGLYGLYGLTHRILETRPGLYGLYLEASPTRPSERPRGLGLGLTYRSERCYSGLYTHRLYSLYSLELYSASNASNTHRARGLEVALGLYGLASNARGLYSGLYGLYLESERPRPRGLNILETHRASNTYRGLYARGILEILESERPHEGLYLYSASPALAALATHRGLNHISSERSERLYSILEALAARGILEASPPHEARGASPALAVALLYSGLYSERHISPRGLYASNHISCYSASPILETRPTHRGLTYRGLNGLILEGLYVALGLYGLYILELYSALAVALTHRASPTYRLYSVALTYRTHRALASERSERVALLEASPMETLEASPPHEVALALAPRLYSLEMETALAARGGLYASNALAHISPHESERHISGLYILEALAASPASNLEASPASPPRLYSTYRGLNHISPHELYSTYRTRPSERASNPRLEGLTHRLYSLEPRASNALAPRASPMETGLILETYRSERLETYRGLYVALGLYILEPRTHRGLARGALATYRVALTYRLYSGLNTHRSERALAGLYGLCYSALAILEPRPHEGLNILEASPALASERALAGLYASPASPSERGLARGSERCYSLELYSGLYGLYVALLESERVALASNGLYASPGLTHRVALPRVALILESERALAGLYPHEMETSERALALYSGLYTRPARGGLYLYSTHRARGPHEASNPRSERGLYILEARGTHRHISILEARGGLTYRASPHISALAPRPRALATHRLELEGLGLYARGGLYTHRGLNSERGLYALAHISVALASPILEMETGLYASNPHEALALEILEGLASPILEILEARGVALALAALAGLYALASERGLYGLASPLEGLYGLYASPARGVALTYRSERASPILEPHELYSTRPSERASPLYSILELYSLEARGLE

SEQ ID NO: 12 - protein, 1601 aa, Linum usitatissimum
METSERPRGLYILEVALTHRGLYGLYLEGLLETRPGLGLYLYSALACYSALALYSASPLEPHEARGLYSARGLETRPGLYGLYTHRPHETHRASPVALLETHRARGPRLECYSTRPLEGLHISLESERLEASPASNGLNTHRGLYLEASPPRPRGLYILEARGVALARGALAVALTHRGLYLEVALSERALAASPTYRPHEALAPRGLYTYRPHEVALTRPALAVALLEILEGLASNLEALALYSILEGLYTYRGLGLYLYSASNLEHISMETALAALATYRASPTRPARGLESERPHEHISASNTHRGLILEARGASPGLNSERLESERARGLELYSSERLYSILEGLGLNMETTYRALASERASNGLYASNLYSLYSVALVALVALVALPRHISSERMETGLYGLYVALTYRPHELEHISPHELELYSTRPVALGLTHRPRTHRPRILEGLYGLYGLYGLYGLYPRASPTRPCYSSERLYSHISILELYSALAVALMETASNILEGLYPRALAPHELEGLYVALPRLYSTHRVALSERASNPHEPHESERALAGLALALYSASPVALALALEILEARGALAMETASPPRGLYLELEASPSERGLMETLEGLYLEGLNTHRLEGLHISVALLELYSVALSERARGTHRTRPASPSERVALVALSERLELEPRLYSGLYGLYGLALAILETRPGLYASPLEASPTRPSERPRGLASPASNASNTYRALASERCYSGLYASPLESERLYSARGLYSALALEGLNSERPRASPASPGLNASNSERTHRSERGLYGLYASPALATHRTHRPHELELELYSGLPRILELYSPHEGLYARGLEILESERPHEGLYLYSALAALASERGLLEPRSERSERLELESERSERLEASPGLNLYSASPLEILELESERGLTHRLYSASNSERSERTHRLESERCYSLYSASPILELYSTHRGLTYRASPGLMETASNARGGLYILEILEPRLYSILEALAGLASNLYSALATYRTHRALATHRTHRVALLEASPLELEARGVALVALALAPRLYSTHRMETLYSARGALAGLALAHISPHESERHISGLYILEALAASPASNLEASPASPPRLYSTYRTHRHISTYRLYSTYRTRPSERASNPRLEGLTHRLYSLEPRASPALAPRASPMETGLILETYRCYSLETYRGLYVALGLYILEPRTHRGLARGSERTYRVALTYRLYSMETSERPRASNTHRARGCYSASNSERILEPRPHEARGILEASPSERSERVALVALGLYASPASPGLYASPSERCYSLEARGGLYGLYVALTYRHISVALASPGLYASPGLSERVALPRVALLESERALAGLYPHEMETCYSALALYSGLYTRPLYSGLYARGTHRARGPHEASNPRSERGLYILESERSERTYRVALARGGLTYRGLNHISLYSPRPRALASERLELEGLGLYARGGLYALAGLSERGLYALAHISVALASPILEMETGLYASNVALGLYLEILEGLASPVALMETARGVALALAALAGLYALATHRGLYSERGLILEGLYGLYASPARGILEHISSERASPILELEARGMETSERASPARGILEASNILEGLNLE

SEQ ID NO: 13 - DNA, 24 bp, Artificial Sequence
  oligonucleotide forward primer LuPDAT1
atgtctctcttgaggcggaggtgg

SEQ ID NO: 14 - DNA, 24 bp, Artificial Sequence
  oligonucleotide reverse primer LuPDAT1
ttaaagccgcaacttaatcttgtc

SEQ ID NO: 15 - DNA, 45 bp, Artificial Sequence
  oligonucleotide forward primer LuPDAT2
atatggtacctacacaatgtcagtagtccgccgccgaaaaccttc

SEQ ID NO: 16 - DNA, 39 bp, Artificial Sequence
  oligonucleotide reverse primer LuPDAT2
atataagcttctagagattcagtttgatcctatcagacc

SEQ ID NO: 17 - DNA, 44 bp, Artificial Sequence
  oligonucleotide forward primer LuPDAT6
atatggtacctacacaatgtccccaggaatcgtcaccggcggtc

SEQ ID NO: 18 - DNA, 38 bp, Artificial Sequence
  oligonucleotide reverse primer LuPDAT6
atatctcgagtcacagttgaatgttaatacggtccgac

SEQ ID NO: 19 - DNA, 32 bp, Artificial Sequence
  forward primer LuFAD2-1
ataggatccaccatgggtgctggtggaagaat

SEQ ID NO: 20 - DNA, 30 bp, Artificial Sequence
  reverse primer LuFAD2-1
tatggtacctcacagcttgttgttgtacca

SEQ ID NO: 21 - DNA, 42 bp, Artificial Sequence
  forward primer LuFAD3B
ccggaattctacacaatgtcaatgagccctccaaactcaatg

SEQ ID NO: 22 - DNA, 29 bp, Artificial Sequence
  reverse primer LuFAD3B
tatgagctctcagctggatttggacttgg

SEQ ID NO: 23 - DNA, 48 bp, Artificial Sequence
  forward pimer LuFAD2-FAD3
gagaggcggtttgcgtattgggcgcgctgaattggagcgacctcatgc

SEQ ID NO: 24 - DNA, 49 bp, Artificial Sequence
  reverse primer LuFAD2-FAD3
gtcagtgagcgaggaagcggaagactggatcttcgagcgtcccaaaacc

SEQ ID NO: 25 - DNA, 49 bp, Artificial Sequence
  forward primer pYES
ggttttgggacgctcgaagatccagtcttccgcttcctcgctcactgac

SEQ ID NO: 26 - DNA, 48 bp, Artificial Sequence
  reverse primer pYES
gcatgaggtcgctccaattcagcgcgcccaatacgcaaaccgcctctc

SEQ ID NO: 27 - DNA, 40 bp, Artificial Sequence
  forward primer LuPDAT1
tataaagctttacacaatgtcactcttgaggcggaggtgg

SEQ ID NO: 28 - DNA, 36 bp, Artificial Sequence
  reverse primer LuPDAT1
tataggatccttaaagccgcaacttaatcttgtcag

SEQ ID NO: 29 - DNA, 42 bp, Artificial Sequence
  forward primer LuPDAT2
atatctcgagtacacaatgtcggtagtccgccgccgaaaacc

SEQ ID NO: 30 - DNA, 39 bp, Artificial Sequence
  reverse primer LuPDAT2
tatatctagactagagattcagcttgatcctatcagacc

SEQ ID NO: 31 - DNA, 37 bp, Artificial Sequence
  forward primer LuPDAT6
tatacccgggtaaacaatgtcgcctggaatcgtcacc

SEQ ID NO: 32 - DNA, 36 bp, Artificial Sequence
  reverse primer LuPDAT6
tatacccgggtcacagttgaatgttaatacggtccg